(19) United States
(12) Patent Application Publication
(10) Pub. No.: US 2022/0176171 A1
(43) Pub. Date: Jun. 9, 2022

(54) FACEPIECE RESPIRATOR

(71) Applicant: University of Connecticut, Farmington, CT (US)

(72) Inventors: Thanh Nguyen, South Windsor, CT (US); Eli Curry, Franklin, CT (US); Thinh T. Le, Willington, CT (US); Thi Tra Nguyen, Storrs, CT (US)

(21) Appl. No.: 17/541,658

(22) Filed: Dec. 3, 2021

Related U.S. Application Data
(60) Provisional application No. 63/120,846, filed on Dec. 3, 2020.

(51) Int. Cl. A62B 23/02; A61L 2/10; D04H 1/728

(52) U.S. Cl. CPC A62B 23/025; A61L 2/10; D04H 1/728; A61L 2202/11; B82Y 40/00; D10B 2331/04; D10B 2401/041; D10B 2401/12; D10B 2509/00; A61L 2202/26

(57) ABSTRACT

A facepiece respirator configured to be worn by a user. The facepiece respirator includes including a first layer and a second layer coupled to the first layer. The first layer is positionable adjacent the user. A filtering layer is positioned between the first and second layer, and the filtering layer includes a piezoelectric film.

### CROSS-REFERENCE TO RELATED APPLICATIONS

The present application is a non-provisional of and claims benefit of U.S. Provisional Patent Application No. 63/120,846, filed on Dec. 3, 2020, the entire contents of which are incorporated herein by reference.

### BACKGROUND

Facepiece respirators or masks (e.g., surgical masks that are FDA approved and thus authorized for use in medical applications) and non-medical masks (e.g., surgical masks that are not FDA approved and thus not authorized for use in medical applications) may be worn by a user over his or her nose and mouth and are configured to filter particulates to prevent the user from inhaling or otherwise ingesting the particulates. Conventional respirators are intended for single use and made from non-degradable materials, causing a serious concern for a plastic-waste environmental crisis. That is, conventional facepiece respirators such as an N95 respirator, a KN95 respirator, and other like surgical masks are made typically from polypropylene, which is a non-degradable and non-piezoelectric material. These respirators are not reusable and are usually disposed of after 8-12 hours of use. Since they are made from a non-degradable material, they will take years to decompose in a landfill, which can cause pollution and harm to the environment. Furthermore, these face masks are weakened in humid conditions and difficult to decontaminate.

The World Health Organization (WHO) declared that conventional face masks such as the N95 and surgical masks are crucial personal protective equipment (PPE) to prevent and suppress the transmission of highly infectious viruses during a pandemic. This fact has also been supported by numerous scientific data. At the beginning of the COVID-19 crisis, high demand along with a shortage of face masks significantly impacted healthcare workers and the general population by leaving them unprotected from the SARS-COV-2 virus. Although COVID-19 infection rates are dropping in developed countries thanks to the vaccination effort, there is still a lack of high-quality face masks for protecting healthcare workers and other consumers due to supply-chain issues. Furthermore, due to the emergence of the highly contagious Delta variant, the current number of COVID-19 cases are rapidly increasing throughout the United States as well as the world. As a result, many countries, especially developing countries, still recommend or even require their citizens to wear face masks in public areas. This global enforcement of face masks has led to billions of N95/surgical masks being disposed of in landfills, causing a significant burden on the environment. It is expected that even after the pandemic, people will still rely heavily on face masks to protect themselves against future infectious viruses. Thus, the trash from one-time use non-degradable face masks will continue to build up, potentially causing an environmental crisis.

Aside from COVID-19, particulate matter (PM), which originates from the combustion of fossil fuels, rapid industrialization, and urbanization around the world, has become a recent topic of interest due to its severe impact on nature and human health. PM is categorized based on its aerodynamic diameter, and the smaller the particles are, the more poisonous and harmful they are to human health since they can travel into deeper parts of the respiratory tract. Specifically, compared to PM 10-coarse particles (diameter from 2.5-10 μm), PM 2.5-fine particles (diameter within 1 to 2.5 μm) are more lethal and are associated with millions of deaths annually. Furthermore, ultrafine particles with a diameter below 1.0 μm (PM 1.0) are the most hazardous and can penetrate directly through the lungs into the bloodstream, imposing a higher risk of lung cancer, as well as cardiovascular diseases when compared to other PMs. Due to such significant health issues, face masks have also been one of the most effective solutions in protecting public health from industrial particles and air pollution. If we continue to rely on traditional nondegradable, disposable face masks, such a high demand for particle-filtering will significantly worsen the environmental issue of non-degradable wastes, and therefore it is necessary to develop an effective air filtration system with reusability and long-term biodegradability, to avoid harm on the environment.

### SUMMARY

Herein is disclosed a reusable, self-sustaining, highly effective, and humidity-resistant air filtration membrane (e.g., film) with excellent particle-removal efficiency, based on highly controllable and stable piezoelectric electrospun PLLA nanofibers. The electrospun PLLA nanofiber filter possesses a high filtration efficiency (>99% for PM 2.5 and >91% for PM 1.0) while providing a favorable pressure drop (˜91 Pa at normal breathing rate) for human breathing due to the piezoelectric charge naturally activated by human respiration through the mask. The filter has a long, stable filtration performance (over 8 weeks) and good humidity resistance, demonstrated by a minimal declination in the filtration performance of the nanofiber membrane after exposure to moisture. The PLLA filter is also reusable via common sterilization processes (i.e., using an ultrasonic cleaning bath, autoclave, or microwave). Moreover, a prototype of a completely biodegradable PLLA nanofiber-based face mask was fabricated and shown to decompose within 5 weeks in an accelerated degradation environment.

In one embodiment, a facepiece respirator is configured to be worn by a user. The facepiece respirator including a first layer and a second layer coupled to the first layer. The first layer is positionable adjacent the user. A filtering layer is positioned between the first and second layer, the filtering layer including a piezoelectric film.

In another embodiment, a facepiece respirator is configured to be worn by a user and including a first layer and a second layer coupled to the first layer, the first layer being positionable adjacent the user, and a filtering layer positioned between the first and second layer, the filtering layer including an electrospun piezoelectric film, wherein each of the first layer, second layer, and filtering layer is formed from sterilizable and biodegradable materials.

In another embodiment, a method of manufacturing a facepiece respirator includes forming a piezoelectric film by electrospinning a first material solution dissolved in a solvent to form a nanofiber mat, applying heat to the nanofiber mat, and positioning the piezoelectric film between a first layer and a second layer, the first layer being positionable adjacent the user.

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an exploded view of a facepiece respirator according to one embodiment including a first layer, a second layer, and a third layer.

FIG. 2A is a plan view of an exemplary third layer for use with the facepiece respirator of FIG. 1.

FIG. 2B is a detailed view of the third layer of FIG. 2A.

FIG. 2C is another detailed view of the third layer of FIG. 2A.

FIG. 2D is a detailed view of an exemplary first layer or second layer for use with the facepiece respirator of FIG. 1.

FIG. 2E is a detailed view of the first layer or second layer of FIG. 2D in combination with an exemplary third layer for use with the facepiece respirator of FIG. 1.

FIG. 2F illustrates the effect that either the first or second layer has on filtering efficiency of the third layer.

FIG. 2G is a detailed view of an exemplary third layer for use with the facepiece respirator of FIG. 1. The scale bars are 500 μm.

FIG. 2H is a detailed view of the first layer or second layer of FIG. 2D in combination with an exemplary third layer including the third layer of FIG. 2G for use with the facepiece respirator of FIG. 1.

FIG. 3A is an exemplary method of manufacture of the facepiece respirators disclosed herein.

FIG. 3B is another exemplary method of manufacture of the facepiece respirators disclosed herein.

FIG. 3C is another exemplary method of manufacture of the facepiece respirators disclosed herein.

FIG. 4A is a table representing the particulate trapping efficiency of the facepiece respirator of FIG. 1 under normal breathing conditions.

FIG. 4B is a table representing the particulate trapping efficiency of the facepiece respirator of FIG. 1 under exerted breathing conditions.

FIG. 5A is a graph representing trapping efficiency and pressure drop under normal breathing conditions of a third layer that is formed at 4000 rpm drum speed using different applied electrical fields.

FIG. 5B is a graph representing trapping efficiency and pressure drop under normal breathing conditions of a third layer that is formed at different drum speeds using a 14 kV electrical field.

FIG. 6A are detailed views of third layers after different periods of exposure to autoclave.

FIG. 6B shows velocity profiles of the third layers of FIG. 6a after each of the different periods of exposure.

FIG. 6C shows a velocity profile of a third layer after autoclave relative to a baseline velocity profile.

FIG. 7A is a schematic view of an exemplary third layer for use with the facepiece respirator of FIG. 1 relative to particulate matter (PM).

FIG. 7B illustrates the collaborative effect of mechanical sieving and piezoelectrically-enhanced electrostatic adsorption in trapping particles in an exemplary third layer for use with the facepiece respirator of FIG. 1.

FIG. 7C illustrates scanning electron microscopy (SEM) images demonstrating the PMs trapping capability of the piezoelectric PLLA nanofibers of an exemplary third layer for use with the facepiece respirator of FIG. 1 before and after filtering. The scale bars for these images are 10 μm.

FIG. 8A illustrates the surface potential of a third layer for use with the facepiece respirator of FIG. 1 compared to commercially available facemask filters.

FIG. 8B illustrates a schematic diagram of the experimental set up for characterizing the piezoelectric response of exemplary third layers for use with the facepiece respirator of FIG. 1 under aeolian vibrations.

FIG. 8C illustrates a photograph of the experimental set up for characterizing the piezoelectric response of exemplary third layers for use with the facepiece respirator of FIG. 1 under aeolian vibrations.

FIG. 8D illustrates the open circuit voltage of an exemplary third layer for use with the facepiece respirator of FIG. 1 and a control layer under different aeolian vibrations.

FIG. 9A illustrates a schematic diagram of the experimental set up for testing the filtration performance of exemplary third layers for use with the facepiece respirator of FIG. 1.

FIG. 9B illustrates a photograph of the experimental set up for testing the filtration performance of exemplary third layers for use with the facepiece respirator of FIG. 1.

FIG. 9C compares the filtering efficiency between exemplary third layers for use with the facepiece respirator of FIG. 1 and control non-piezoelectric PDLLA samples at 5.83 cm/s (normal breathing conditions).

FIG. 9D compares the filtering efficiency between exemplary third layers for use with the facepiece respirator of FIG. 1 and control non-piezoelectric PDLLA samples at 14.16 cm/s (exerted breathing conditions).

FIG. 10A illustrates the filtering efficiency measured at a normal breathing rate of exemplary third layers resulting from different rotating speeds during electrospinning and for use with the facepiece respirator of FIG. 1 for different sizes of PM.

FIG. 10B illustrates the pressure drop measured at a normal breathing rate of exemplary third layers resulting from different rotating speeds during electrospinning and for use with the facepiece respirator of FIG. 1.

FIG. 10C illustrates a quality factor measured at a normal breathing rate of exemplary third layers resulting from different rotating speeds during electrospinning and for use with the facepiece respirator of FIG. 1 for different sizes of PM.

FIG. 10D illustrates the filtering efficiency measured at a normal breathing rate of exemplary third layers resulting from different applied electrical fields during electrospinning and for use with the facepiece respirator of FIG. 1 for different sizes of PM.

FIG. 10E illustrates the pressure drop measured at a normal breathing rate of exemplary third layers resulting from different applied electrical fields during electrospinning and for use with the facepiece respirator of FIG. 1.

FIG. 10F illustrates a quality factor measured at a normal breathing rate of exemplary third layers resulting from different applied electrical fields during electrospinning and for use with the facepiece respirator of FIG. 1 for different sizes of PM.

FIG. 11A illustrates the filtering efficiency measured at a normal breathing rate of exemplary third layers resulting from the use of different solvents during electrospinning and for use with the facepiece respirator of FIG. 1 for different sizes of PM.

FIG. 11B illustrates the pressure drop measured at a normal breathing rate of exemplary third layers resulting from the use of different solvents during electrospinning and for use with the facepiece respirator of FIG. 1.

FIG. 11C illustrates a quality factor measured at a normal breathing rate of exemplary third layers resulting from the use of different solvents during electrospinning and for use with the facepiece respirator of FIG. 1 for different sizes of PM.

FIG. 12A illustrates the filtering efficiency measured at a normal breathing rate of exemplary third layers having different configurations and for use with the facepiece respirator of FIG. 1 for different sizes of PM.

FIG. 12B illustrates the pressure drop measured at a normal breathing rate of exemplary third layers having different configurations and for use with the facepiece respirator of FIG. 1 for different sizes of PM.

FIG. 12C illustrates a quality factor measured at a normal breathing rate of exemplary third layers having different configurations and for use with the facepiece respirator of FIG. 1 for different sizes of PM for different sizes of PM.

FIG. 12D illustrates the filtering efficiency measured at a normal breathing rate of exemplary third layers having different configurations and for use with the facepiece respirator of FIG. 1 for different sizes of PM different sizes of PM.

FIG. 12E illustrates the pressure drop measured at a normal breathing rate of exemplary third layers having different configurations and for use with the facepiece respirator of FIG. 1 for different sizes of PM.

FIG. 12F illustrates a quality factor measured at a normal breathing rate of exemplary third layers having different configurations and for use with the facepiece respirator of FIG. 1 for different sizes of PM for different sizes of PM.

FIG. 12G illustrates the filtering efficiency measured at a normal breathing rate of exemplary third layers having different configurations and for use with the facepiece respirator of FIG. 1 for different sizes of PM for different sizes of PM.

FIG. 12H illustrates the pressure drop measured at a normal breathing rate of exemplary third layers having different configurations and for use with the facepiece respirator of FIG. 1 for different sizes of PM.

FIG. 12I illustrates a quality factor measured at a normal breathing rate of exemplary third layers having different configurations and for use with the facepiece respirator of FIG. 1 for different sizes of PM for different sizes of PM.

FIG. 13A illustrates the decline in efficiency measured at a normal breathing rate of a third layer for use with the facepiece respirator of FIG. 1 compared to those of N95 facemasks and surgical masks after exposure to moisture.

FIG. 13B illustrates the long-term performance test measured at a normal breathing rate of an exemplary third layer for use with the facepiece respirator of FIG. 1 for PM 1.0 removal.

FIG. 13C illustrates the filtering efficiency measured at a normal breathing rate of an exemplary third layer having a two-layer structure with holes organized in a 6×6 array and for use with the facepiece respirator of FIG. 1 for different sizes of PM.

FIG. 13D illustrates the pressure drop measured at a normal breathing rate of an exemplary third layer having a two-layer structure with holes organized in a 6×6 array and for use with the facepiece respirator of FIG. 1.

FIG. 13E illustrates a quality factor measured at a normal breathing rate of an exemplary third layer having a two-layer structure with holes organized in a 6×6 array and for use with the facepiece respirator of FIG. 1 for different sizes of PM.

FIG. 13F illustrates the filtering efficiency of an exemplary third layer for use with the facepiece respirator of FIG. 1 for different sizes of PM as compared to surgical masks and N95 respirators before being humidified.

FIG. 13G illustrates the filtering efficiency of an exemplary third layer for use with the facepiece respirator of FIG. 1 for different sizes of PM as compared to surgical masks and N95 respirators after being humidified.

FIG. 13H illustrates the piezoelectric performance via output charge under impact force of an exemplary third layer for use with the facepiece respirator of FIG. 1 as compared to a control non-piezoelectric layer before being humidified.

FIG. 13I illustrates the piezoelectric performance via output charge under impact force of an exemplary third layer for use with the facepiece respirator of FIG. 1 as compared to a control non-piezoelectric layer after being humidified.

FIG. 13J illustrates the piezoelectric performance via displacement under an applied electrical field of an exemplary third layer for use with the facepiece respirator of FIG. 1 as compared to a control non-piezoelectric layer before being humidified.

FIG. 13K illustrates the piezoelectric performance via displacement under an applied electrical field of an exemplary third layer for use with the facepiece respirator of FIG. 1 as compared to a control non-piezoelectric layer after being humidified.

FIG. 13L illustrates the piezoelectric performance via filtering efficiency of an exemplary third layer for use with the facepiece respirator of FIG. 1 for different sizes of PM as compared to a control non-piezoelectric layer before being humidified.

FIG. 13M illustrates the piezoelectric performance via filtering efficiency of an exemplary third layer for use with the facepiece respirator of FIG. 1 for different sizes of PM as compared to a control non-piezoelectric layer after being humidified.

FIG. 13N illustrates the piezoelectric performance via output charge of an impact test of an exemplary third layer for use with the facepiece respirator of FIG. 1.

FIG. 13O illustrates the piezoelectric performance via output charge of an impact test of an exemplary third layer that was treated with the annealing process and for use with the facepiece respirator of FIG. 1 after 0, 2, and 4 weeks.

FIG. 13P illustrates the piezoelectric performance via output charge of an impact test of an exemplary third layer that was not treated with the annealing process and for use with the facepiece respirator of FIG. 1 after 0, 2, and 4 weeks.

FIG. 13Q illustrates the H NMR spectra of exemplary electrospun third layers made from DCM/DMF solvent dissolved in Chloroform (CDCl3) and for use with the facepiece respirator of FIG. 1.

FIG. 13R illustrates the H NMR spectra of exemplary electrospun third layers made from Chloroform solvent dissolved in deuterated DCM (CD2Cl2) and for use with the facepiece respirator of FIG. 1.

FIG. 13S illustrates a comparison of the mechanical properties (via a tensile test) of exemplary treated third layers for use with the facepiece respirator of FIG. 1 over time.

FIG. 14A illustrates photographs showing the experimental setup using LB agar plates for demonstrating the bacteria retention ability of an exemplary third layer for use with the facepiece respirator of FIG. 1.

FIG. 14B illustrates the LB agar plates that were positioned under an exemplary first or second layer for use with the facepiece respirator of FIG. 1 (Plate A), a control layer (Plate B), an exemplary third layer for use with the facepiece respirator of FIG. 1 (Plate C), and an N95 respirator (Plate D) after 24 hours of incubation at 37 degrees Celsius.

FIG. 14C illustrates photographs showing the experimental setup using testing areas for demonstrating the bacteria trapping capacity an exemplary third layer for use with the facepiece respirator of FIG. 1 and an N95 respirator.

FIG. 14D illustrates the colony forming unit (CFU) count of bacteria at the three selected areas identified in FIG. 14C.

FIG. 15A illustrates a schematic diagram identifying the decontamination of the PLLA air filter via common approaches.

FIG. 15B illustrates the effect of the use of an exemplary third layer for use with the facepiece respirator of FIG. 1 and ultrasound stimulation in lysing a first bacteria.

FIG. 15C illustrates the effect of the use of an exemplary third layer for use with the facepiece respirator of FIG. 1 and ultrasound stimulation in lysing a second bacteria.

FIG. 15D illustrate SEM images of portions of an exemplary third layer for use with the facepiece respirator of FIG. 1 after autoclave cycles. The scale bars are 100 μm.

FIG. 15E compares the filtering efficiencies of exemplary third layers for use with the facepiece respirator of FIG. 1 before and after several cycles of autoclaving.

FIG. 15F illustrate SEM images of portions of an exemplary third layer for use with the facepiece respirator of FIG. 1 after microwaving. The scale bars are 100 μm.

FIG. 15G illustrate fluorescence images of live/dead assay of ADSC cells seeded on an exemplary third layer for use with the facepiece respirator of FIG. 1 before and after microwaving. The scale bars are 500 μm.

FIG. 15H compares the filtering efficiency of exemplary third layers for use with the facepiece respirator of FIG. 1 before and after microwaving.

FIG. 15I compares the filtering efficiency (via live cell count) of the exemplary third layers for use with the facepiece respirator of FIG. 1 before and after microwaving.

FIG. 15J compares the filtering efficiency (via dead cell count) of the exemplary third layers for use with the facepiece respirator of FIG. 1 before and after microwaving.

FIG. 15K illustrates the effect of exemplary third layers for use with the facepiece respirator of FIG. 1 and ultrasound on Indigo Carmine dye in PBS 1λ, pH 7.4 buffer.

FIG. 15L illustrates the detection of the hydroxyl radical by using HPF dye (hydroxyl radical sensor) of exemplary third layers for use with the facepiece respirator of FIG. 1 with and without ultrasound as well as with ultrasound alone.

FIG. 15M illustrates the detection of the singlet oxygen sensor by using singlet oxygen sensor green of exemplary third layers for use with the facepiece respirator of FIG. 1 with and without ultrasound as well as with ultrasound alone.

FIG. 15N illustrates the electron paramagnetic resonance spectra (EPR) of radical spin-trapped by DMPO for different exemplary third layers for use with the facepiece respirator of FIG. 1 with and without ultrasound as well as with ultrasound alone in aqueous (PBS) dispersion, which indicates detection of the hydroxyl radical.

FIG. 15O illustrates the electron paramagnetic resonance spectra (EPR) of radical spin-trapped by DMPO for different exemplary third layers for use with the facepiece respirator of FIG. 1 with and without ultrasound as well as with ultrasound alone in aqueous (PBS) dispersion, which indicates the detection of the super oxide anion in dimethyl sulfoxide (DMSO) dispersion.

FIG. 15P illustrates the stability of exemplary third layers for use with the facepiece respirator of FIG. 1 before and after ultrasound sonification process using FTIR spectra.

FIG. 15Q illustrates the mechanical stability of exemplary third layers for use with the facepiece respirator of FIG. 1 before and after ultrasound sonication process.

FIG. 15R compares the mechanical properties of exemplary third layers for use with the facepiece respirator of FIG. 1 after different cycles of autoclaving.

FIG. 15S compares average velocity profiles of exemplary third layers for use with the facepiece respirator of FIG. 1 during actuation after different periods of exposure to an autoclave.

FIG. 15T illustrates that if the exemplary third layer for use with the facepiece respirator of FIG. 1 is damaged or broken down, the velocity profiles will be minimal.

FIG. 15U compares the pressure drop values of exemplary third layers for use with the facepiece respirator of FIG. 1 after different cycles of autoclaving.

FIG. 15V compares the pressure drop values of exemplary third layers for use with the facepiece respirator of FIG. 1 after microwaving for various periods of time.

FIG. 15W compares the mechanical properties of exemplary third layers for use with the facepiece respirator of FIG. 1 after microwaving for various periods of time.

FIG. 16A illustrates an exemplary facepiece respirator.

FIG. 16B illustrates the degradation of an exemplary facepiece respirator in an accelerated degradation condition.

FIG. 16C compares the mechanical properties of an exemplary third layer for use with the facepiece respirator of FIG. 1 with surgical mask.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

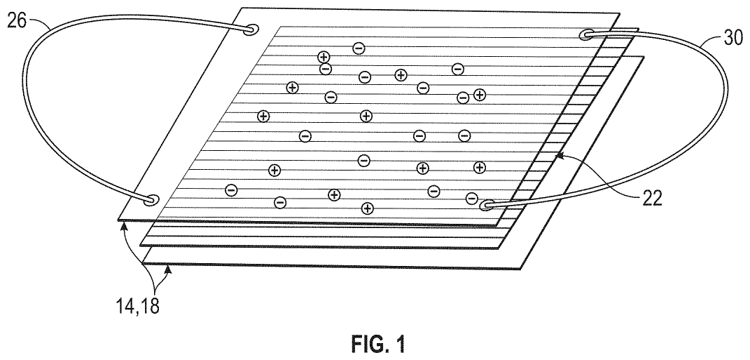

FIG. 1 illustrates a facepiece respirator or mask 10 including a first layer 14, a second layer 18 coupled to the first layer 14, and a third layer 22. Either the first layer 14 or the second layer 18 defines a surface that is positionable adjacent to the nose and mouth of the user. The third or filtering layer 22 is positioned between the first and second layers 14, 18. In other or additional embodiments, the mask 10 may include only the first layer or the second layer in combination with the filtering layer 22. A first loop 26 is coupled to a first end of the mask 10 and configured to receive an ear of the user and a second loop 30 coupled to a second, opposite end of the body and configured to receive another ear of the user. In other or additional embodiments, the first loop 26 and the second loop 30 may extend from the first end to the second end. In still other or additional embodiments, a single loop may extend from the first end to the second end. The first layer 14, second layer 18, first loop 26, and the second loop 30 are formed from a reusable, biocompatible, and biodegradable medical-grade material. Exemplary materials for the first layer, second layer, first loop, and the second loop include, but are not limited to, poly-(l-lactic acid) (PLLA), poly(D, L-lactic acid) (PDLLA), polycaprolactone (PCL), poly (lactic acid) (PLA), poly (lactic-co-glycolic acid) (PLGA), polyurethane (PUR), polyhydroxybutyrate (PHB), chitin, chitosan, natural rubber, cotton, and/or a combination thereof. In the illustrated embodiments, the first and second layers 14, 18 are formed from PLLA and the first and second loops are formed from a combination of natural rubber and cotton. In some embodiments, the first and second loops may also include wire.

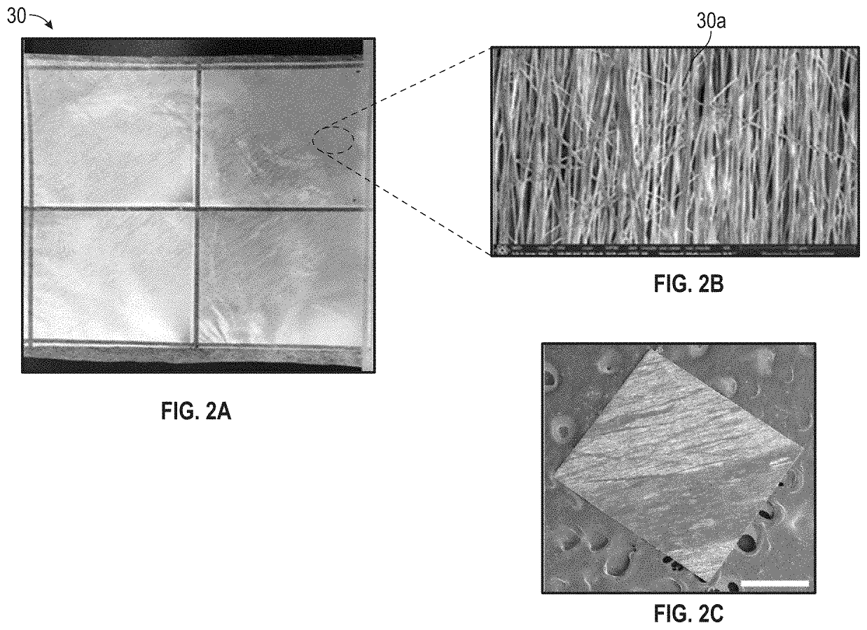

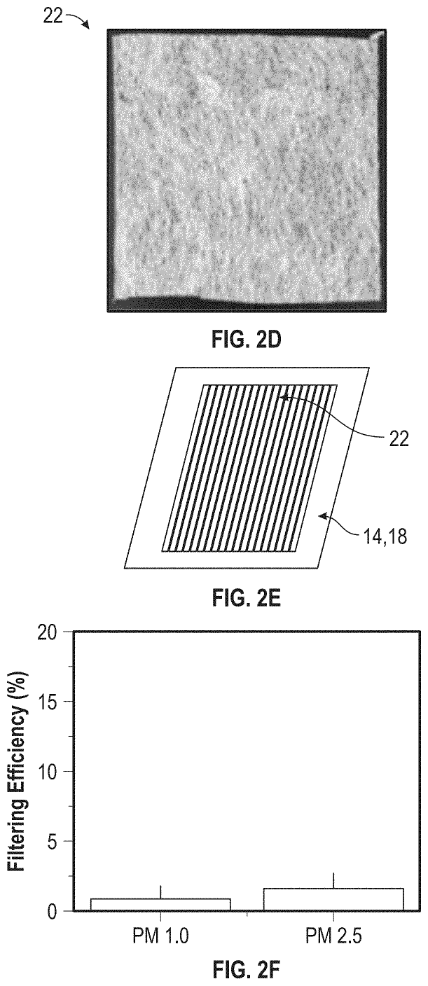

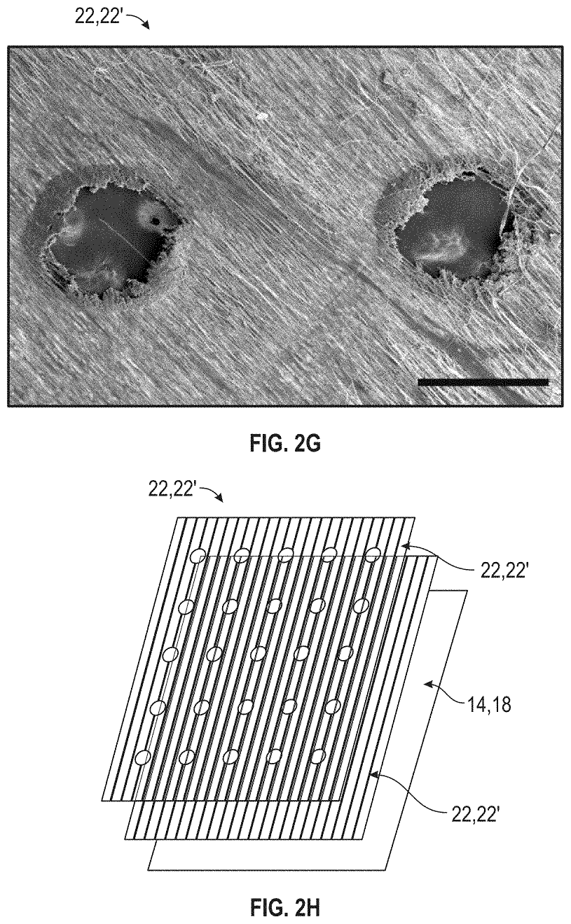

FIGS. 2A-2H illustrate examples of the filtering layer 22 in greater detail. The filtering layer 22 includes a reusable, biocompatible, and biodegradable medical-grade material. In the illustrated embodiments, the filtering layer 22 is a piezoelectric film (e.g., also may be referred to herein as a piezoelectric membrane, a PLLA filter, a PLLA nanofiber membrane, a PLLA nanofiber filter, or a PLLA nanofiber film) formed from nanofibers 30a formed using poly-(l-lactic acid) (PLLA). In other embodiments, the piezoelectric film may be formed from nanofibers 30a formed from other exemplary materials such as, but not limited to, poly-(l-lactic acid) (PLLA), poly-(d-lactic acid) (PDLA), polycaprolactone (PCL), chitosan, chitin, poly(γ-benzyl-L-glutamate) (PBLG) or their combination with nanofiller dopants such as amino acid crystals (e.g. glycine) or nanocellulose. More specifically, the filtering layer 22 is formed from a heat-treated PLLA nanofiber mat including nanofibers 30a. The film exhibits piezoelectricity, which efficiently filters small particles (e.g., bacteria and viruses). The piezoelectricity from PLLA nanofibers 30a may enhance their trapping efficiency when compared to using the inherent surface charge of the PLLA alone. In the illustrated embodiment, as shown in the scanning electron microscope (SEM) image of FIG. 2C, the PLLA nanofiber mat is cut at a 45 degree angle relative to the oriented direction of the PLLA nanofibers 30a, which is 0 degrees or 180 degrees in FIG. 2C. The scale bar of FIG. 2C is 500 μm, although the dimensions of the film used for the image of FIG. 2C are not to scale for films used as the final filtering layer 22. Moreover, in the illustrated embodiment and as shown in the SEM image of FIG. 2B, the nanofibers 30a of PLLA nanofiber mat have a diameter of approximately 300 nm.

The process for manufacturing the filtering layer 22, which will be discussed in greater detail below, enables the characteristics of the filtering layer 22 to be modified or tuned to meet different needs or goals. That is, porosity, fiber orientation, molecular weight, crystallinity or nanofiller dopant (e.g. glycine crystals, nanocellulose, etc.) of the nanofibers can be adjusted during manufacturing to adjust the piezoelectricity, filter efficiency, and lifetime of the piezoelectric film. Accordingly, in other or additional embodiments the thickness of the nanofiber may range from approximately 100 nm to 1 μm. “Approximately” as used herein means plus or minus 100 nm.

In other embodiments, for example, shown in FIG. 2G, the filtering layer 22 may further include one or more holes. In still other embodiments, shown in FIG. 2H, the filtering layer 22 may be a multi-layer filtering layer including more than one PLLA nanofiber mat including nanofibers 30a. In the embodiment, shown in FIG. 2H, one of the multi-layer filtering layers 22′ includes a first PLLA nanofiber membrane having holes and a second PLLA nanofiber membrane 22″ that does not have holes. In the illustrated embodiments, the holes of the filtering layers 22 are circular holes having a dimension (e.g., a diameter) of approximately 500 nanometers. In other embodiments, the holes may have other shapes and dimensions. That is, the holes may be triangular, rectangular, square, polygonal, etc. Moreover, the holes may have a dimension of 300 micrometers to 700 micrometers. The holes may also be formed in a variety of arrays (e.g., 4×4, 5×5, 6×6, 12×12, etc.). Additionally, in other embodiments, there may be additional PLLA nanofiber mats, with or without holes, included in the filtering layer 22.

Importantly, the facepiece respirator 10 described herein is reusable because it is capable of being sterilized. That is, the facepiece respirator 10 is capable of being decontaminated one or more times via an autoclave, an ultrasonic, or a sonication (e.g., with saltwater) cleaning bath. After several sterilization cycles the piezoelectricity of the material will decrease, at which point the face mask can be thrown away to decompose in a landfill, resulting in no harm to the environment. As noted above, the lifetime of the facepiece respirator 10 may be modified via the manufacturing process.

The facepiece respirator 10 is biodegradable, reusable and made of a commonly-used medical-grade polymer that utilizes the piezoelectric effect to enhance the mask's filtering efficiency and offers the ability to be disinfected. The facepiece respirator 10 is designed to be especially helpful in protecting users (e.g., healthcare professionals, patients, first responders, teachers, people with underlying health conditions, or any other individual) against the SARS-CoV-2 virus, among other potentially harmful bacteria and viruses. In particular, the facepiece respirator 10 may be used in lieu of known non-biodegradable and single-use facepiece respirators, such as a N95 respirator, a KN95 respirator, and other like surgical masks, which may be in short-supply.

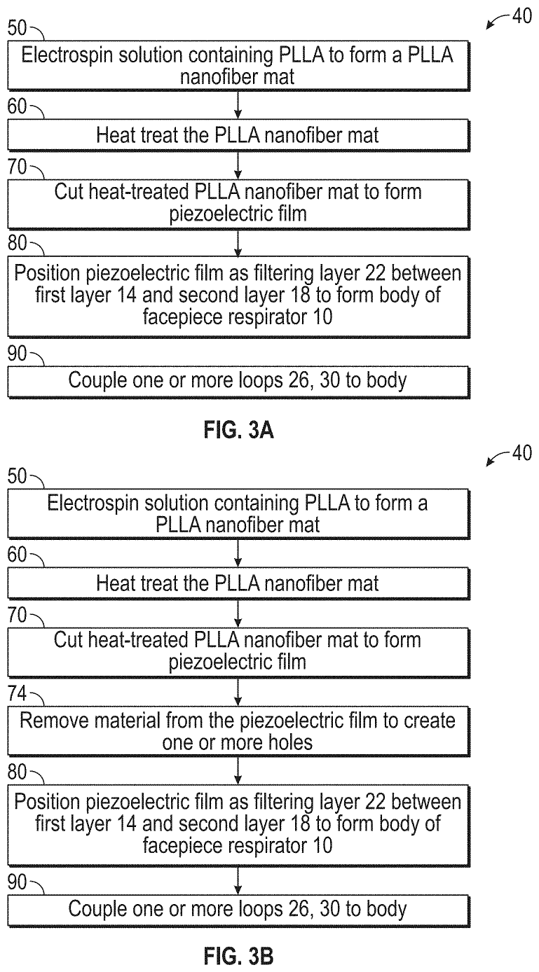

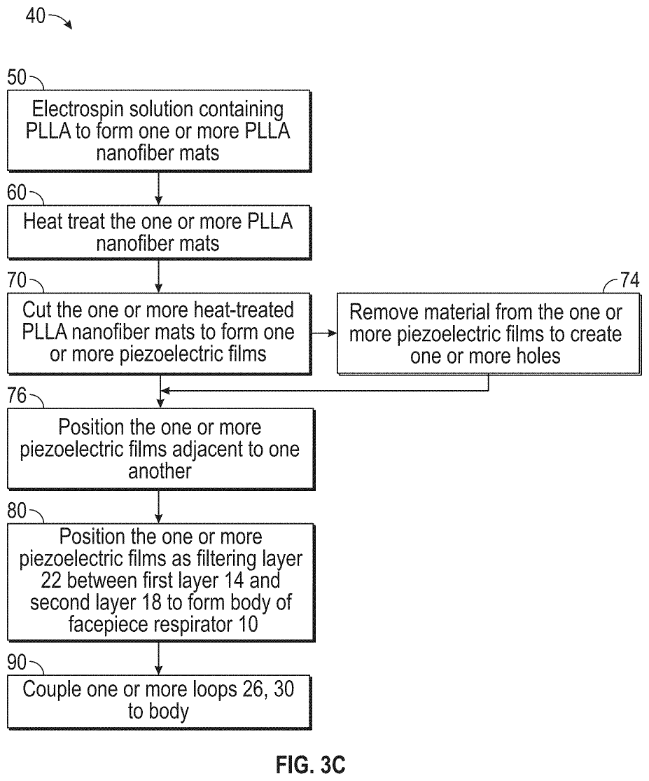

With respect to FIG. 3A, the method of manufacturing 40 is disclosed. The facepiece respirator 10 is manufactured by forming the piezoelectric film of the filtering layer 22 by electrospinning a solution containing PLLA to form a PLLA nanofiber mat 50, heat treating (e.g., applying heat to) the PLLA nanofiber mat 60, and cutting the heat-treated nanofiber mat 70 to form the piezoelectric film. In some embodiments (FIGS. 3B and 3C), at step 74, the method further includes removing (e.g., via laser cutting) material of the piezoelectric film to create holes extending therethrough. In some embodiments (FIG. 3C), at step 76, the method further includes positioning multiple piezoelectric films adjacent to one another to create a multi-layer filtering layer 22.

The electrospinning process includes preparing a solution of PLLA in a solvent. In one embodiment, the process included preparing a solution of a 4% w/v of PLLA dissolved in a 1:4 mixture of N,N-Dimethylformamide (DMF), and dichloromethane (DCM). In another embodiment, the process included preparing a solution of 0.8 g of PLLA dissolved in 20 ml of Chloroform solvent (4% w/v).

Electrical voltage is then applied to the prepared solution. In the illustrated embodiment, the prepared solution is pumped at a constant rate of 2-3 ml/hr through a needle with a high DC voltage applied (e.g. 14-30 kV) applied to it. The needle size can vary from 18G to 23G and, its tip can either be flat or sharp. The change in the needle parameters and the DC voltage amplitude will vary the fiber morphology and topology, as well as the fiber mats thickness which offer the ability to tune the filter performance of the nanofiber mats. The resulting electrified solution is then collected on a drum that rotates at a predetermined rotation per minute (rpm). In the illustrated embodiment the electrified solution is sprayed at a grounded aluminum drum rotating at speeds from 300 rpm-4,500 rpm to create the nanofiber mat of PLLA. The rotating speed of the drum can be adjusted to modify the fiber thickness and the alignment of the nanofibers over the entire film (180 degree+/−10 degrees). For example, a PLLA nanofiber mat may have nanofibers with a thickness of approximately 600-700 nm when the drum is spun at 3,000 rpm and having median alignment (70-80% of fibers aligned). When the drum is spun at 300 rpm the resulting PLLA nanofiber mat will have nanofibers measuring approximately 800 nm to 1 μm and having very low fiber alignments (15-20% of fibers aligned) whereas when the drum is spun at 4,000 rpm the resulting PLLA nanofiber mat will have nanofibers measuring approximately 300-500 nm and high fiber alignment over the entire fiber mat (90-95% of fibers aligned).

The PLLA nanofiber mat is heat treated by a two-step annealing process. In particular, the PLLA nanofiber mat is annealed at a first temperature for a period of time and then allowed to cool to room temperature. Then, subsequently, the PLLA nanofiber mat was annealed at a second temperature for another period of time and then, again, allowed to cool to room temperature. In the illustrated embodiments the first temperature is 105 degrees Celsius and the second temperature is 160.1 degrees Celsius. Although the first and second temperatures are different in the illustrated embodiments, in other or alternative embodiments the first and second temperatures may be the same. Moreover, the first and second temperatures may be greater than or less than those stated above. The temperature can fluctuate from 75 degrees Celsius to 110 degrees Celsius and 153 degrees Celsius to 165 degrees Celsius for the first time and second time of annealing, respectively. In the illustrated embodiment, the first period of time and the second period of time is 10 hours. Although the first and second periods of time are the same in the illustrated embodiments, in other or alternative embodiments the first and second periods of time may be different. Moreover, the first and second periods of time may be greater than or less than 10 hours (e.g., 1 hr to 15 hrs.).

As noted above, the resulting heat-treated PLLA nanofiber mat is then cut to form the piezoelectric film. Specifically, and with renewed reference to FIGS. 2C-2E and 2G-2H, the heat-treated PLLA nanofiber mat is cut at a 45-degree angle relative to the direction the fibers are oriented, which is considered to be 0 degrees or 180 degrees, in order to harvest the shear piezoelectric signal and form the piezoelectric film. At element 80, the piezoelectric film positioned the first layer and the second layer as the filtering layer 22 to form the body of the facepiece respirator 10. The first and second layers are supporting layers. The first, second, and third layers 14, 18, 22 are coupled to one another (e.g., by ultrasonic welding and an impulse heat sealer) to form the body. At element 90, the first and second loops are coupled to the body (e.g., by ultrasonic welding and an impulse heat sealer) to the form the facepiece respirator 10 to complete the facepiece respirator 10.

As noted above, the piezoelectricity and pore size of the PLLA nanofiber film can be tuned by engineering the electrospinning parameters (e.g., electric field, drum rotation speed, etc.), as reported in Curry, Eli J., et al. “Biodegradable nanofiber-based piezoelectric transducer.” Proceedings of the National Academy of Sciences 117.1 (2020): 214-220, which is incorporated herein by reference.

Experiments, Results, and Discussion

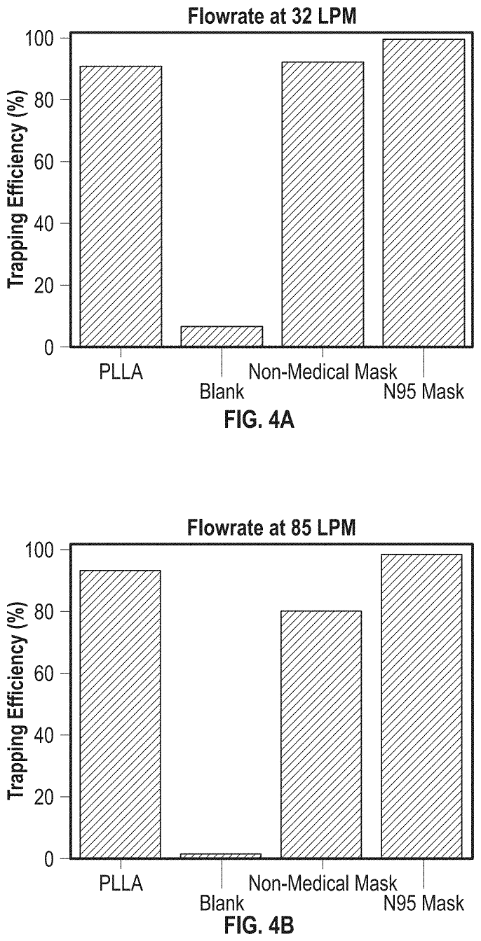

As shown in FIGS. 4A and 4B, in one experiment, the trapping efficiency of the facepiece respirator 10 was tested by using the TSI8130A instrument under different flow rates 32 L/min (which represents normal breathing) and 85 L/min (which represents breathing under exertion) at an age of 25, in accordance with National Institute for Occupational Safety and Health (NIOSH) protocols. For the facepiece respirator 10 described above manufactured using the method described above having an area measuring 15 cm by 15 cm, the trapping efficiency is about 90% at normal breathing rates and 93% under extreme conditions. The pressure drop of air through the facepiece respirator 10 may be adjusted via adjustment of the pore sizes of the PLLA nanofiber mat.

As noted above, changing the electrospinning parameters such as needles (e.g., needle size and tip shape), DC voltage amplitude, and rotating speed of the collector will vary the fibers' morphology, topology, alignments as well as the thickness of the piezoelectric film. Accordingly, the filter performance of the piezoelectric film may be tuned based on one or more of these parameters. In one example, the thickness of the piezoelectric film may account for the trapping efficiency of a facepiece respirator 10. That is, a thicker piezoelectric film may result in a facepiece respirator 10 with enhanced trapping efficiency but reduced breathability due to the increase in the pressure drop. On the other hand, a thinner piezoelectric film results in a facepiece respirator 10 with enhanced breathability but a reduced trapping efficiency. Also, if the piezoelectric film is too thin, it will be fragile and make the facepiece respirator fabrication process difficult. This issue can be overcome by assembling multiple piezoelectric films to achieve an appropriate thickness for the filter layer, which provides a high trapping efficiency (above 90%) and an acceptable pressure drop value (350 Pa at 85 LPM). That is, the filtering layer 22 may include one or more piezoelectric films.

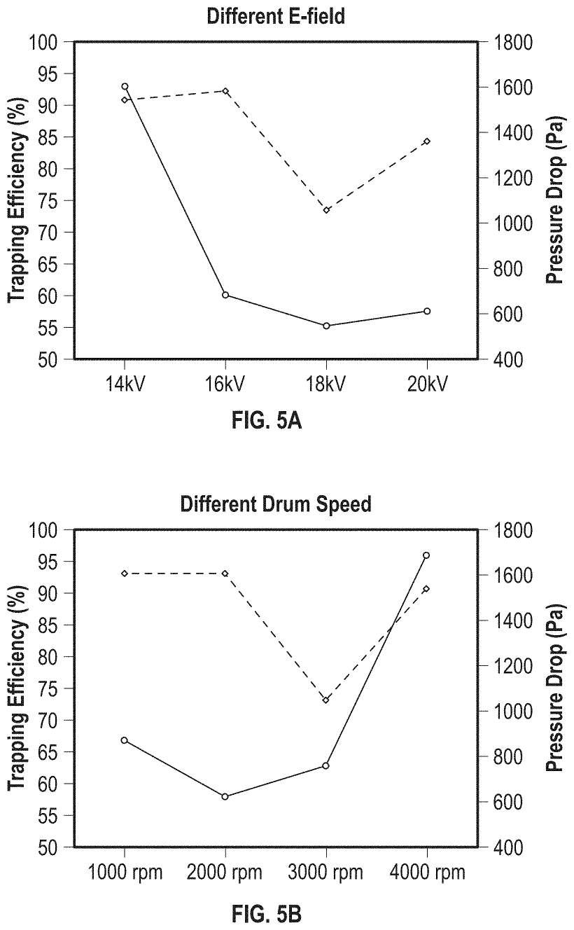

The ability to tune the piezoelectric film is demonstrated by FIGS. 5A and 5B. In particular, several piezoelectric films were electrospun at different applied electrical field amplitudes (FIG. 5A) and different drum rotation speed (FIG. 5B). Then, the trapping efficiency and pressure drop of the resulting piezoelectric film were measured at normal breathing conditions (32 L/min, i.e., LPM).

With respect to FIG. 5A, a first piezoelectric film was electrospun at 4000 rpm and 14 kV, which resulted in a trapping efficiency of nearly 95% and a pressure drop of around 1600 Pa under normal breathing conditions. A second piezoelectric film was electrospun at 4000 rpm and 16 kV, a third piezoelectric film was electrospun at 4000 rpm and 18 kV, and a fourth piezoelectric film was electrospun at 4000 rpm and 20 kV. As shown in FIG. 5A, the second, third, and fourth piezoelectric films have better breathability but less trapping efficiency than the first piezoelectric film. Accordingly, varying the applied electrical field amplitude can vary the efficacy of the facepiece respirator 10.

With respect to FIG. 5B, a first piezoelectric film was electrospun at 1000 rpm and 14 kV, which resulted in a trapping efficiency of nearly 95% and a pressure drop of over 800 Pa under normal breathing conditions. A second piezoelectric film was electrospun at 2000 rpm and 14 kV, which resulted in a trapping efficiency of nearly 95% but a pressure drop of around 600 Pa. A third piezoelectric film was electrospun at 3000 rpm and 14 kV, which resulted in a trapping efficiency of nearly 75% but a pressure drop of below 800 Pa. A fourth piezoelectric film was electrospun at 4000 rpm and 14 kV, which resulted in a trapping efficiency of around 95% and a pressure drop of over 1600 Pa. The first and second piezoelectric films resulted in at least the same or better trapping efficiency and better breathability than that of the fourth piezoelectric films. Accordingly, varying the drum speed can vary the efficacy of the facepiece respirator 10.

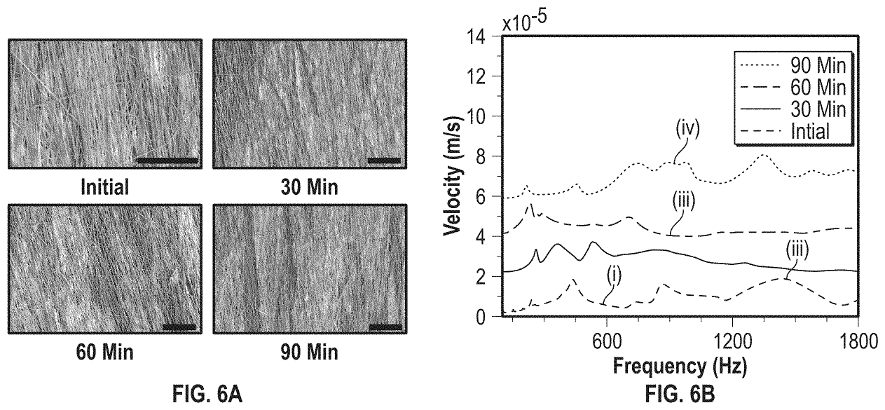

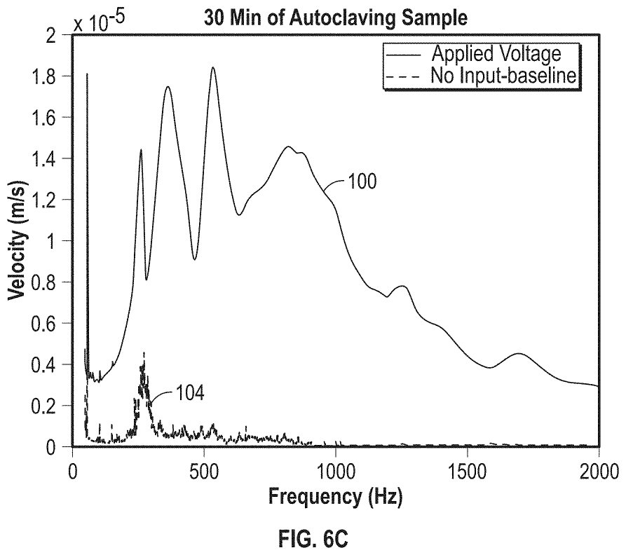

In order to illustrate the reusability of the facepiece respirator 10, several piezoelectric films cut from the same PLLA nanofiber mat were autoclaved for varying periods of time. Because the piezoelectric films of FIG. 6A were cut from the same PLLA nanofiber mat, their properties (e.g., the fibers morphology, topology, alignments and overall mat thickness) should be substantially the same. FIG. 6A shows various SEM images (having a scale bar of 100 μm) of the autoclaved piezoelectric films. As shown, image (i) shows an SEM image of a first piezoelectric film without autoclaving, image (ii) shows an SEM image of a second piezoelectric film after being autoclaved for one 30-minute cycle (e.g., 30 minutes total), image (iii) shows an SEM image of a third piezoelectric film after being autoclaved for two 30-minute cycles (e.g., 60 minutes total), and image (iv) shows an SEM image of a fourth piezoelectric film after being autoclaved for three 30-minute cycles (e.g., 90 minutes total). All of the second, third, and fourth autoclaved piezoelectric films maintained piezoelectric properties after subjected to autoclaving for various amounts of time. In particular, as shown in the SEM images of FIG. 6A, the macroscopic piezoelectric films remained intact after three periods of exposure to the autoclave and the piezoelectric films still exhibited piezoelectric properties. While it can be seen that the autoclave begins to break down the fourth piezoelectric film after a total of 90 minutes exposure (FIG. 6A), the filtering layer 22 may still be utilized for the piezoelectric effect (FIG. 6B).

The post-autoclave piezoelectric properties of the various piezoelectric films are demonstrated by the velocity profiles of FIG. 6B. The velocity profiles of FIG. 6B were obtained using a vibrometer system, which utilizes the Doppler effect to measure the vibration, to test the conversed piezoelectric effect of the piezoelectric films. The velocity profiles of FIG. 6B show that piezoelectric films still exhibit piezoelectricity after being autoclaved. The pattern of the velocity profiles represents how fast the piezoelectric films vibrate at different frequencies. If the piezoelectric film is damaged or broken down, the velocity profiles will be minimal. As shown in FIG. 6B, the peak-to-peak magnitude of the velocity of piezoelectric film samples of FIG. 6A are comparable. The mechanical properties, morphology, and topology of the piezoelectric films may slightly change due to autoclave, which is why their velocity profiles pattern are slightly shifted. Also there are some differences in the velocity profiles amongst the different piezoelectric films, which may be attributed to differences in thickness, ultimate composition, or mechanical properties. Generally, with respect to FIG. 6C, the velocity profile of piezoelectric film 100 with a minimal piezoelectricity should have the same profile as the velocity profile of the piezoelectric films under no electrical input 104, may be considered as a baseline.

FIGS. 6A and 6B demonstrate that the piezoelectric film, and therefore the facepiece respirator 10, may be autoclaved for at least 90 minutes and maintain their piezoelectric properties.

Filtration Mechanism and Fabrication of the Piezoelectric PLLA Nanofiber-based Air Filter

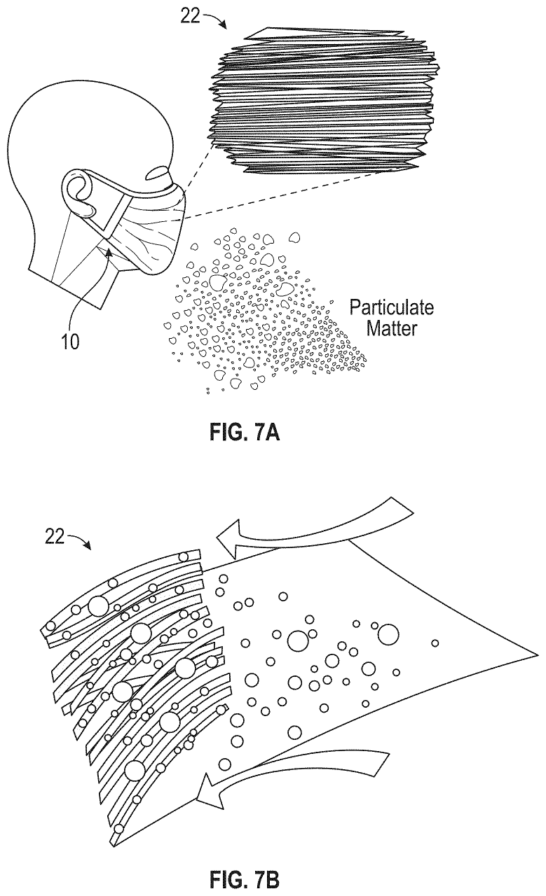

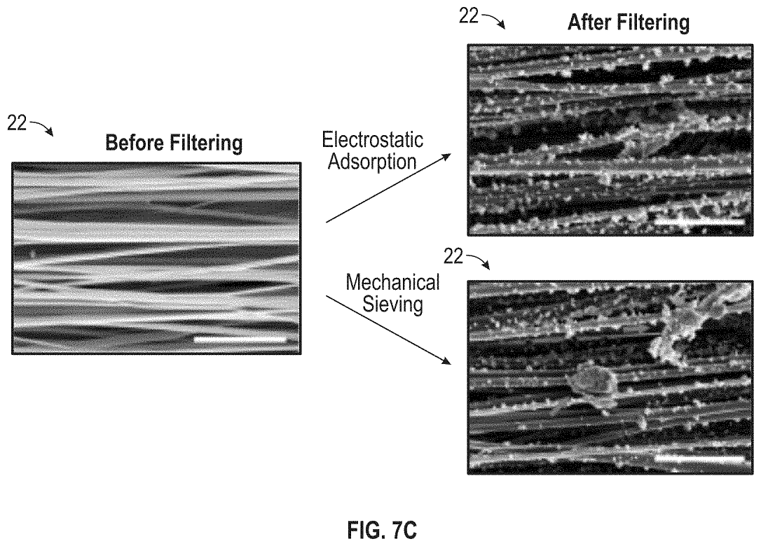

As is discussed throughout, the PLLA electrospun nanofiber mat is used as the air filtering material because of its vigorous and flexible mechanical properties, thermal stability, biodegradability, and especially, piezoelectric response which enhances the efficiency of air-filtering. The piezoelectricity enables the PLLA nanofiber membranes to be self-charged, based on aeolian vibrations, which allow the combined effect of mechanical sieving (from the nanofiber mesh) and the enhanced electrostatic adsorption for the removal of PMs (FIG. 7B), especially for fine and ultrafine particles. This is clearly demonstrated in the scanning electron microscopy (SEM) images of the electrospun PLLA nanofibers for air/particle filtering before and after the filtration process (FIG. 7C). SEM was performed on PLLA samples to visualize the morphology of the PLLA fibers. The arrows indicate coarse particles.

Electrospinning was used to fabricate uniform piezoelectric nanofibers of PLLA. The macroscopic fiber orientation and the molecular alignment (which directly impact the piezoelectric performance of the samples) were tuned by varying the speed of the rotating collector drum and the jet speed (i.e., the electrical field applied during electrospinning).

The same amount of solution (20 ml) was used every time doing electrospinning. The solution flow rate was 2 ml/hr through a flat-tipped 21-gauge needle (Jensen Global, Santa Barbara, Calif.). The distance from the needle tip to the collector drum is kept at 3¾″ (˜8.5 cm). The applied electric field can vary from 12 kV to 20 kV. The polarized solution was then sprayed at a grounded aluminum drum, wrapped in aluminum foil, rotating at speeds from 300-4,000 rpm (rotations per minute). The experiments were conducted in a 30±10% relative humidity atmosphere at ambient temperature. This results in a PLLA nanofiber mat with varying degrees of alignment and fiber morphologies that appear to be based on rotating drum speed and applied electrical field.

After the electrospinning process, the samples were annealed and slowly cooled down to achieve a stable piezoelectric effect which is crucial to maintaining the filtering efficiency of the facemask including the PLLA nanonfiber piezoelectric film. Specifically, these fibrous mats were then annealed at 105° C. for 10 hr, and then the oven (Quincy Lab Inc., Chicago, Ill.) was shut off and allowed to cool to room temperature. The fibrous mats were then peeled off the aluminum foil and transferred to a Teflon FEP sheet (American DURAFILM, Holliston, Mass.). Another Teflon sheet was placed on top of the film, and then the sandwiched film was placed on top of a glass slide. The sandwiched film was then placed inside an oven at 160.1° C. for 10 hr and then the oven was shut off and allowed to cool to room temperature.

Reproducibility is one of the main challenges in fabricating the electrospun PLLA nanofiber film. Therefore, in an attempt to achieve consistency and minimize the deviation from batch to batch of the electrospun nanofiber mats, the electrospinning parameters were strictly controlled. In addition, after electrospinning, the samples' thickness and weight density were characterized to assure their uniformity. As a result, the thicknesses and weight density of the electrospun samples (3 batches made independently) fabricated from the same parameters are comparable (<5% variation in thickness and <9% fluctuation in weight density).

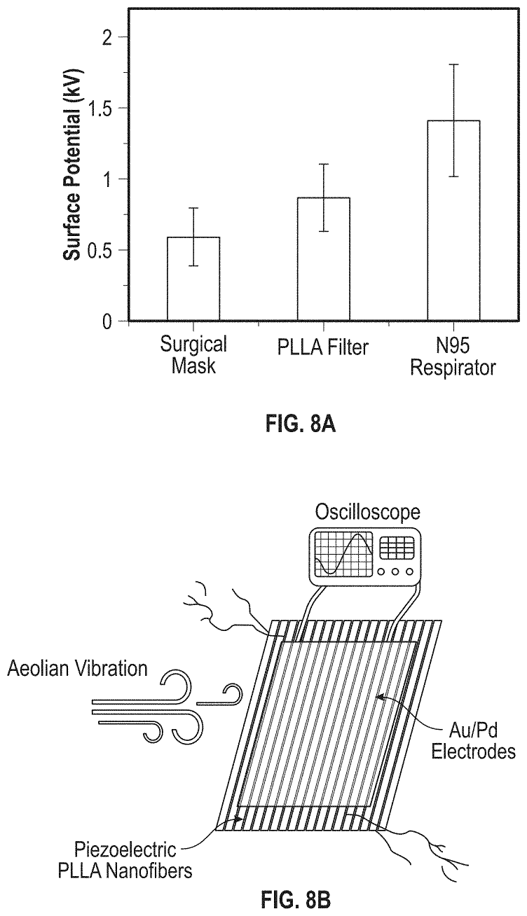

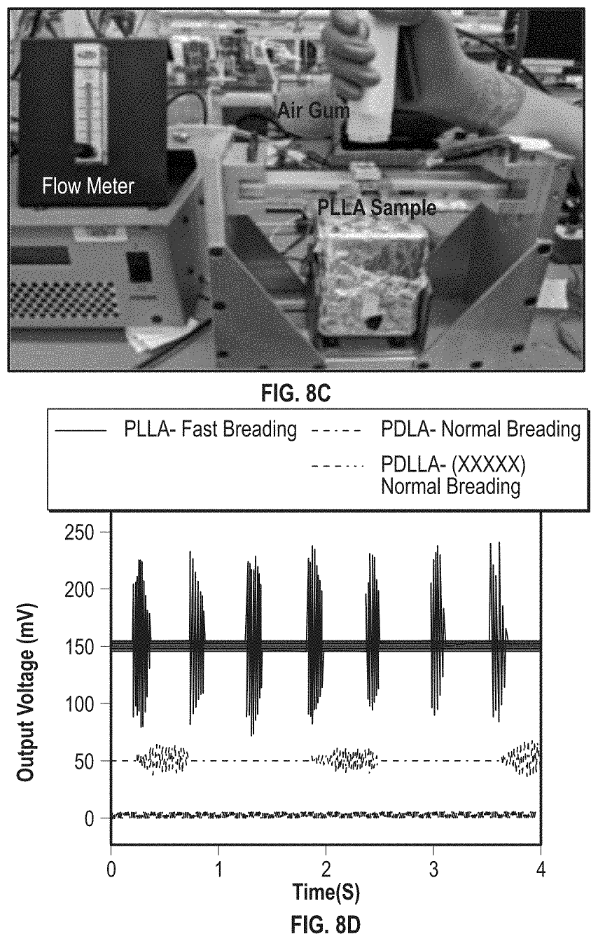

Given that the surface potential is one of the parameters that directly affects the electrostatic adsorption ability of the membrane, a non-contacting electrostatic probe (Trek Inc, 502-1-CE) was used to investigate this value. The surface potential of the electrospun PLLA filter is about 0.9 kV which is greater than the value of the surgical mask filtering layer but smaller than the value of the N95 respirator (1.4 kV) (FIG. 8A). However, it is noteworthy that the electrostatic adsorption of the material will be boosted under aeolian vibrations (i.e., human breathing) due to the piezoelectricity. Thus, the self-charging ability of the piezoelectric PLLA nanofiber film was assessed by measuring the piezoelectric response (i.e., electrical output) of the samples under aeolian vibrations. Gold/palladium (Au/Pd) electrodes were deposited on the PLLA nanofiber film by sputter-coating to collect the electrical signal. The natural breathing of a human was mimicked by utilizing a system comprised of an air gun and a pressure valve/gauge to control the airflow; the electrical output was measured by using a digital oscilloscope (FIG. 8B-C). The samples were exposed to different aeolian vibrations (e.g., 5.83 cm/s and 14.16 cm/s) generated by the air gun. The testing face velocity was designed as 5.83 cm/s, normal breathing rate, and 14.16 cm/s (fast breathing rate), considering this is usually regarded as the industrial testing standard according to the European standard (EN779: 2012) and USA standard (IEST-RP-CC52.2-2007) for air filters.

In addition, to justify the performance of the PLLA piezoelectric nanofibers, a non-piezoelectric negative control of poly(D, L-lactic acid) (PDLLA) nanofiber mat, fabricated by the same electrospinning method, was employed. The PDLLA copolymer has the same chemical components as PLLA, except that its molecular structure has alternating D-lactic and L-lactic acid groups on the backbone, hence eliminating the net polarization of all dipole moments, which makes PDLLA amorphous and non-piezoelectric.

FIG. 8D illustrates the open-circuit voltage output from the PLLA nanofiber membrane under different breathing rates. Remarkable output signals of 150.1 mVpp and 34 mVpp were measured from the piezoelectric PLLA nanofiber film samples at a fast-breathing rate and normal breathing rate, respectively, while the non-piezoelectric PDLLA sample exhibits negligibly small voltage output (˜3 mVpp) under the same magnitude of flow and breathing rate. This result not only validates the piezoelectric response generated by the electrospun PLLA nanofiber membrane under aeolian stimulations but also implies the improvement of the electrostatic adsorption for PM removal at higher airflow, based on the self-charging abilities of piezoelectric PLLA.

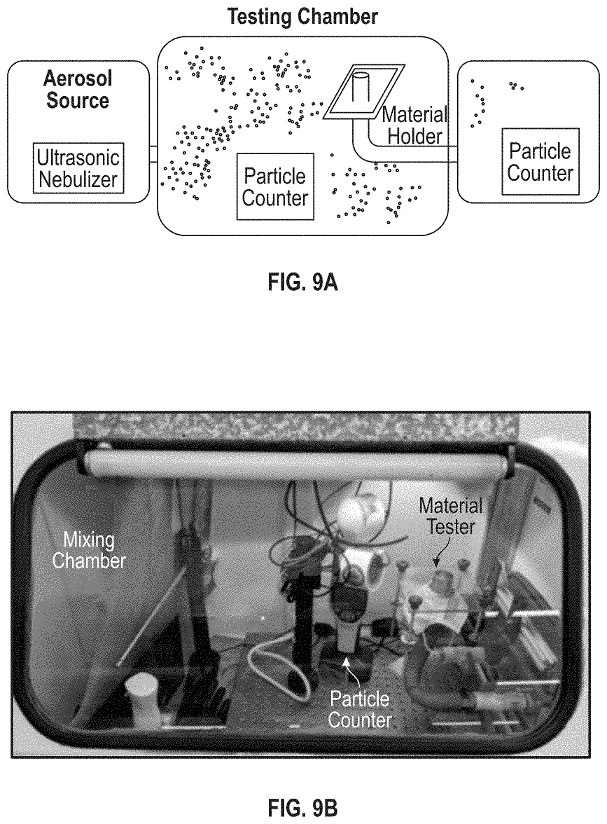

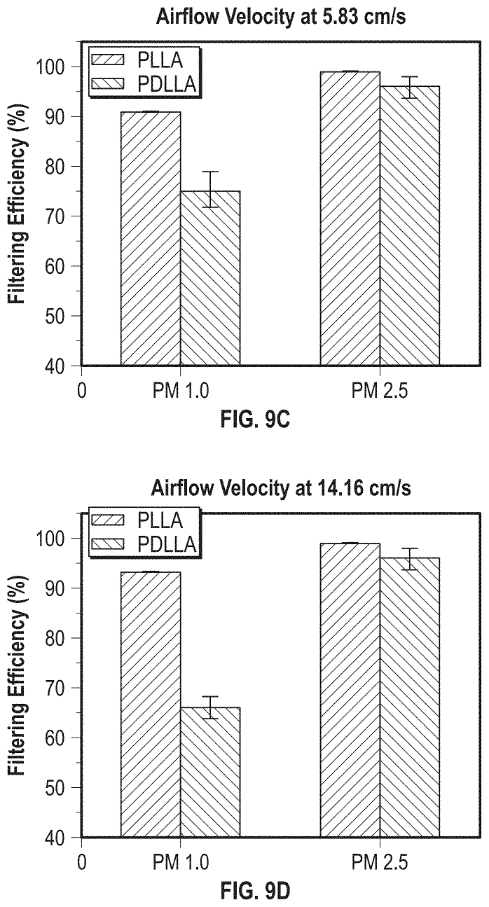

To assess the filtering performance of the piezoelectric PLLA nanofiber film, a custom-built apparatus for rapid comparative filtration testing was employed. The nonpiezoelectric PDLLA nanofiber films were used as a control for this experiment. The schematic and photographs of the system are described in FIGS. 9A-B. Briefly, the apparatus consisted of an aerosol source, a glove box chamber simulating the shared air space before filtering, and a glovebox load-lock chamber representing the environment after filtering. Given that the focus of this work is to prevent the spreading of viruses (e.g., Sars-CoV-2), which are believed to be transmitted by both coarse droplets (˜5 μm in diameter) and fine aerosols (<2.5 μm in diameter), tap water was used as a source of aerosols, producing particle concentration typically 10 μg/m3 in PM 1.0 particle size. A plume of aqueous aerosols was created by pulsing an ultrasonic nebulizer. A gas pump and thermal mass flow meter (TSI 4100, USA) were connected to the testing chamber to control the gas flow through the system. A differential pressure gauge (TSI 9565, MN, USA) was used to monitor the pressure drop. In addition, particle counters were placed on two ends of the testing system to measure the number of particles before and after the filtration.

There is particulate loss as they travel from the testing chamber to the secondary glovebox. For each measurement, the number of particles was measured at the two chambers with the sample and also without the sample (i.e., opened pipe to provide the baseline value). The filtering efficiency value of the testing material was normalized to the baseline values. The quality factor was computed using the following formula:

$$QF = \frac{( - ln ( 1 - E ) )}{Δ P}$$

With E representing the filtering efficiency and ΔP representing the pressure drop across the filter.

As shown in FIG. 9C, the filtering performance (representing the ability to trap the particles) of PLLA is slightly greater than the PDLLA in regard to the removal of PM 2.5 (i.e., particles size from 1-2.5 μm) at a normal breathing rate (airflow velocity at 5.83 cm/s). This is because the large particles can be easily trapped by any nanofiber membrane. However, in terms of PM 1.0 (i.e., fine particles from 0.3-1 μm), the filtering efficiency of PLLA can achieve up to ˜91.5±0.3%, which is significantly greater than that of the PDLLA nanofiber film (˜76.3±3.5%). This difference in filtering efficiency between PM 2.5 and PM 1.0 could be attributed to the filtration mechanism difference at two different particle sizes. To further elaborate, at PM 2.5, the mechanical sieving of the nanofiber membranes mainly contributes to the entrapment of the particles; on the other hand, for PM 1.0, the electrostatic adsorption plays a crucial role in trapping the fine particles as they can easily avoid mechanical interactions with the nanofiber mesh. As a result, the filter made from PLLA outperformed the PDLLA samples due to the reinforced electrostatic charge through the piezoelectric effect. Furthermore, at a fast-breathing rate (airflow velocity at 14.16 cm/s), the difference in the filtering efficiency between the two samples is even more significant-93.8% compared to 65.9% for PM 1.0, due to the greater piezoelectric response of the PLLA (FIG. 9D).

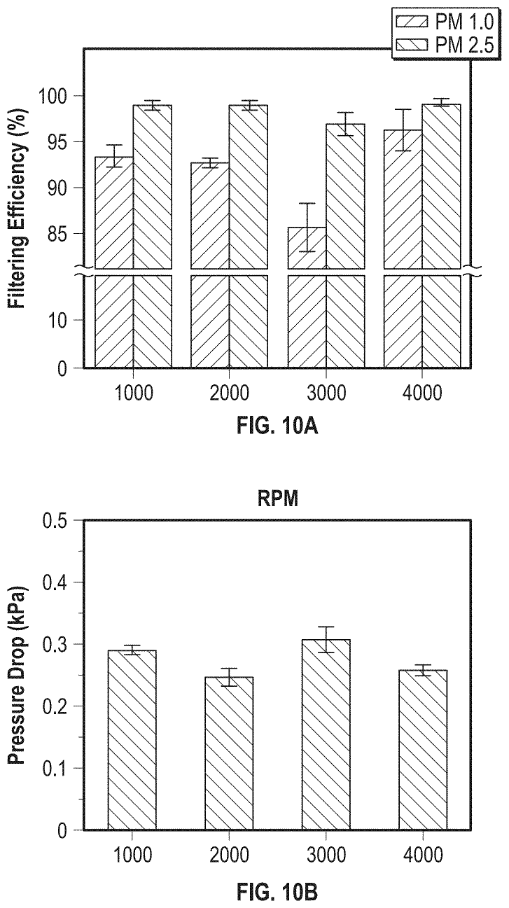

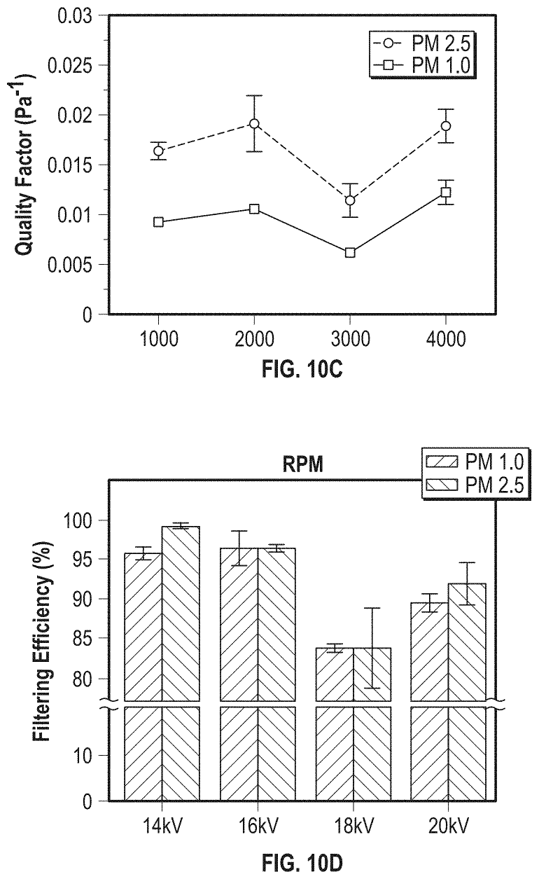

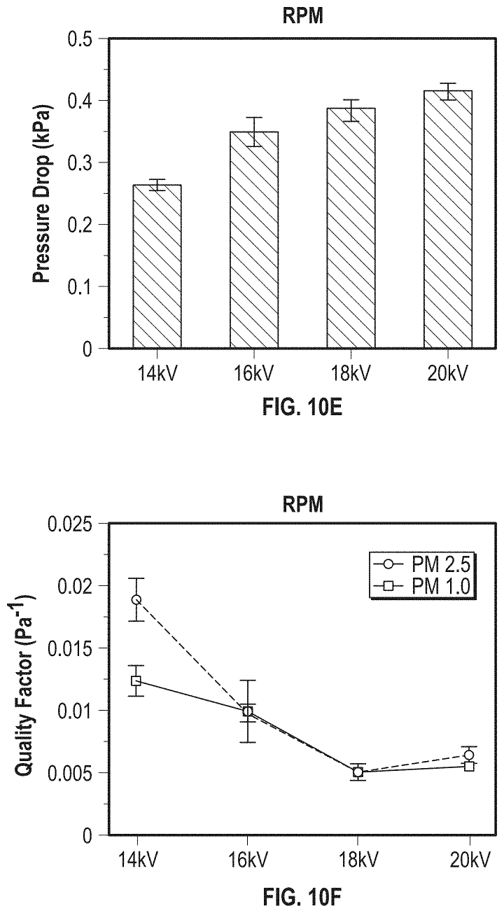

As aforementioned, changing the electrospinning parameters such as the rotating speed of the collector and applied electrical field will vary the fibers morphology, topology, and alignment, which can influence the filtering performance of the PLLA piezoelectric nanofiber film. Hence, after validating the role of piezoelectricity in enhancing electrostatic adsorption, the effect of the electrospinning parameters on the filtration performance of the PLLA nanofiber films was investigated. In this experiment, PLLA samples were fabricated from different electrospinning parameters and assembled on non-woven fabric supporting layers for the filtration test (FIGS. 2D-F). Several indexes, including (1) filtering efficiency, (2) air pressure differential, and (3) quality factor, were assessed to evaluate a facemask's filtration performance. The air pressure drop is associated with breathability, and the quality factor describes the overall filtration performance of the facemask through the balance between the filtering efficiency and the air pressure drop. This quality factor was used as the figure of merit and the main criterion to optimize the electrospinning parameters. It is also worth noting that the thickness of the PLLA samples significantly influences the filtration performance; hence the electrospinning time, the distance from needle to the collector, and polymer solution concentration remained unchanged to make the thicknesses of all single-layer PLLA samples consistent in the range of 30-37 μm. The area density (weight per area—g/m2) is also used as a valid measure to ensure the thicknesses of these samples are comparable to each other. FIGS. 10A-C demonstrate the performance (i.e., filtering efficiency, pressure drop and quality factor) of different PLLA samples, at the airflow velocity of 5.83 cm/s, which were made from different collector drum rotating speeds (1000-4000 rpm). The results show that the electrospun sample collected at 4000 rpm (which has the greatest piezoelectric output) has the largest filtering efficiency at 95.8% and 99.2% for PM 1.0 and PM 2.5, respectively, with a pressure drop of 260 Pa. This result demonstrates significant benefits of the piezoelectric response on enhancing filtering efficiency.

Next, different jet speeds were also investigated by changing the applied electrical field magnitude; the filtration performances of these samples are shown in FIGS. 10D-F. At a higher jet speed, corresponding to greater applied electrical field, the quality factors of these samples are further decreased. Specifically, the quality factor was reduced by more than 60% for PM 2.5 (from the 0.0188 Pa-1 at 14 kV to 0.0063 Pa-1 at 20 kV applied electrical field) and about 50% for PM 1.0 (0.0123 Pa-1 at 14 kV to 0.0055 Pa-1 at 20 kV). As a result, 4,000 rpm and 14 kV applied electrical field were chosen as the optimal parameters for electrospinning the PLLA nanofiber samples.

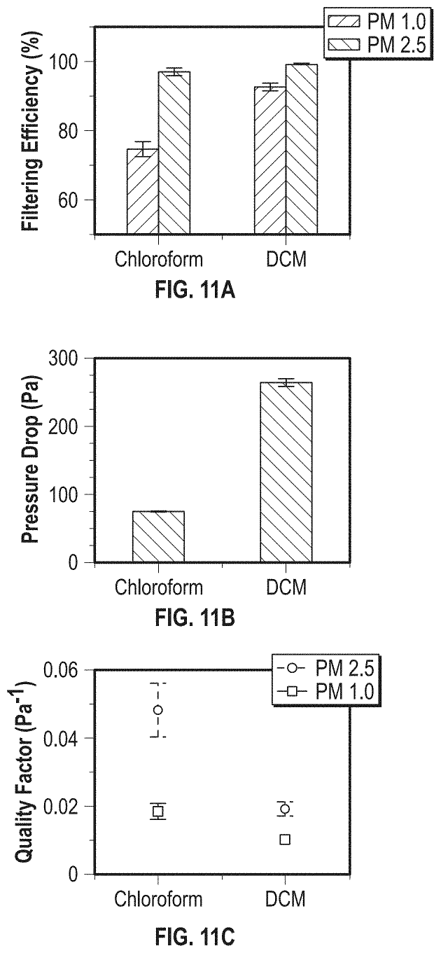

As the pressure differential through the PLLA filters was high (around 260 Pa), which may make the masks difficult to breathe through, several methods were attempted to reduce this value while maintaining the filtering efficiency of the samples. To do so, replacing the solvent used for the electrospinning method (i.e., Dichloromethane (DCM)/Dimethylformamide (DMF)) with Chloroform was investigated. As shown in FIGS. 11A-C, the pressure differential substantially reduced from around 260 Pa to 75 Pa at a normal breathing rate by changing the solvent while keeping other parameters constant. Nevertheless, the filtering efficiency of the samples made from Chloroform also was reduced. Hence, one of the samples was an integrated structure comprised of two layers of piezoelectric PLLA nanofiber film in which the bottom PLLA layer is the original PLLA nanofiber film while the top PLLA layer was patterned with tiny holes (˜500 μm in diameter), by a laser cutter, in order to improve the ventilation (FIGS. 2G-H).

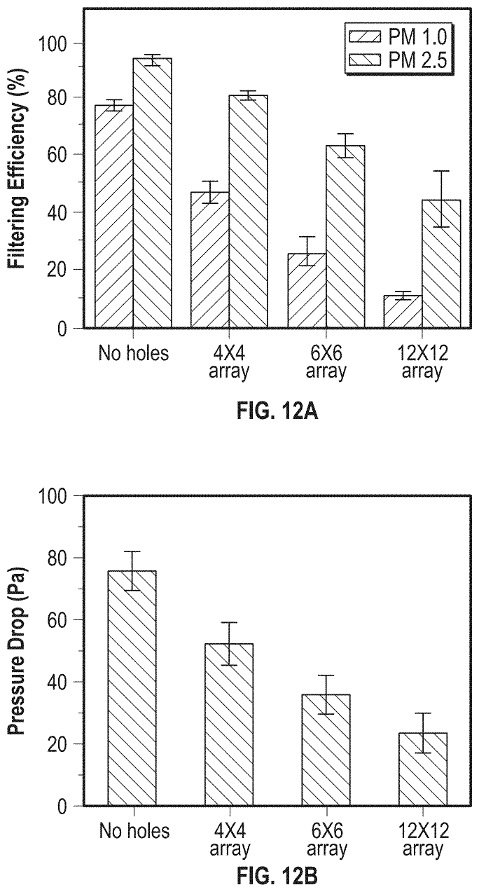

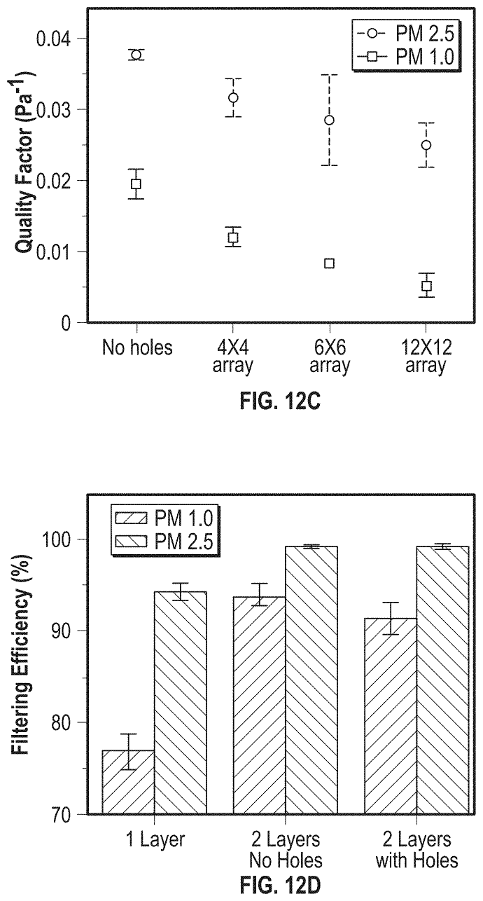

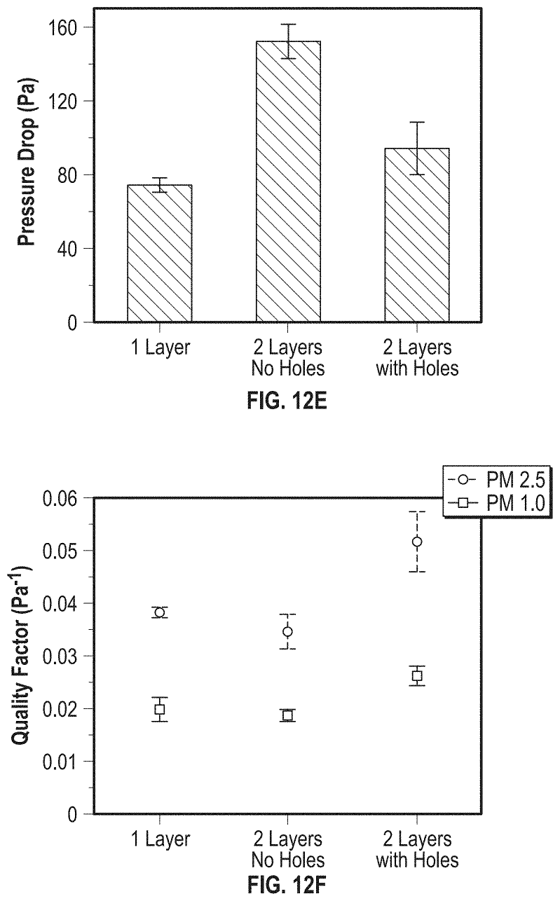

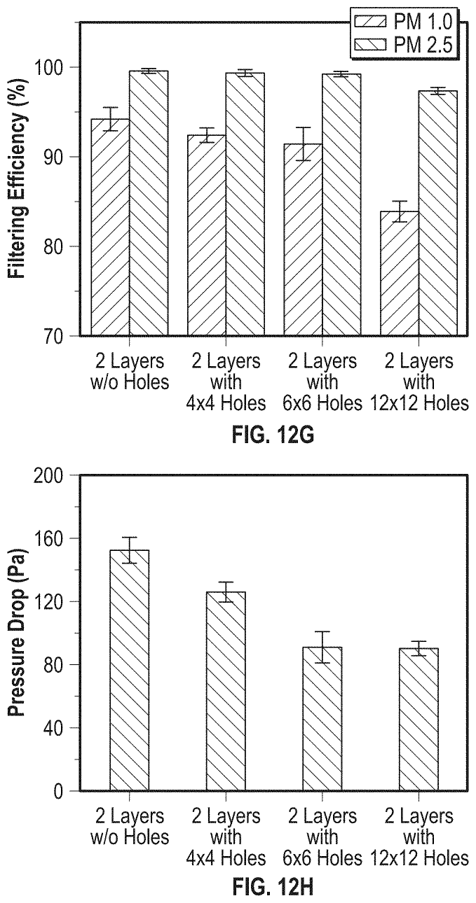

It was then investigated how changing the number of holes affected the masking performance of a PLLA piezoelectric nanofiber film as described in FIGS. 12A-C. It can be seen that as the number of holes increased, the pressure-drop, filtering efficiency, and the quality factor values reduced significantly. However, by assembling a layer of PLLA piezoelectric nanofiber film patterned with holes (6×6 array) on another as-is PLLA piezoelectric nanofiber film, the two-layer structure exhibited a greater filtration performance compared to a single-layer sample as well as a two-layer sample without holes (FIGS. 12D-I). Although the filtering efficiency of the two-layer sample with holes is slightly smaller than the sample without holes (91.5% compared to 94.1% and 99.2% versus 99.5% for PM 1.0 and PM 2.5, respectively), the two-layer sample with holes shows a superiority in terms of pressure drop (91 Pa at the normal breathing rate for the two-layer sample with holes), which results in an exceptional quality factor, compared to the two-layer sample without holes.

Moisture Resistance, Long-Term Stability and Durability of the Filtration Performance of PLLA.

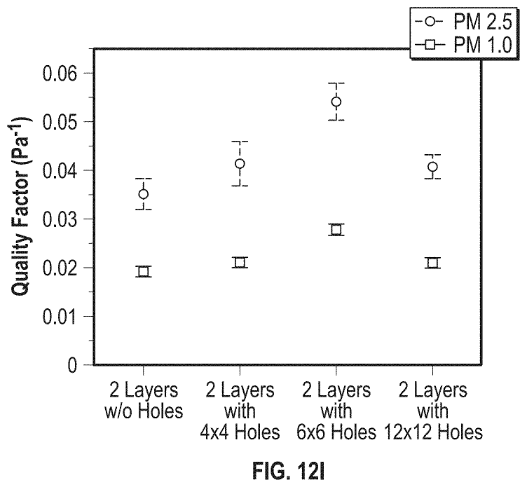

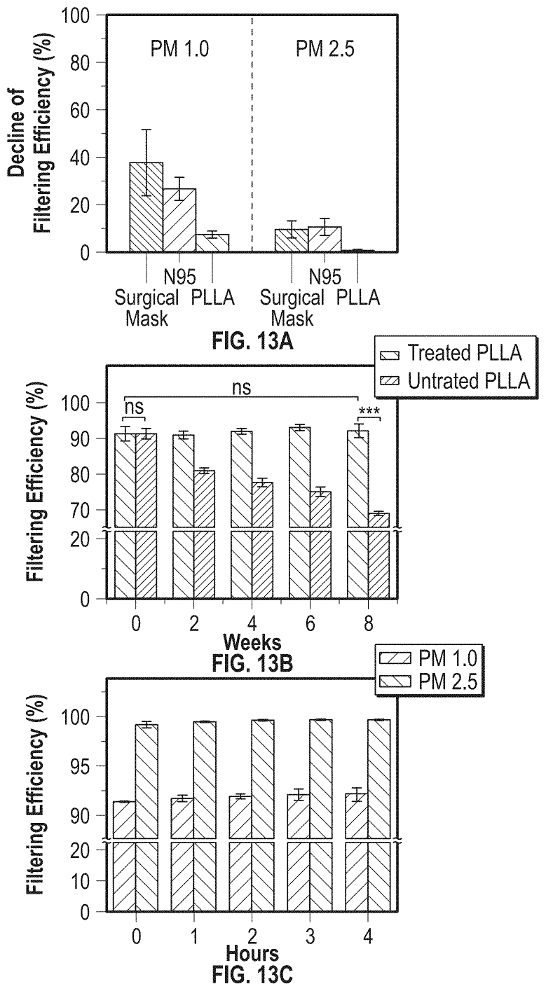

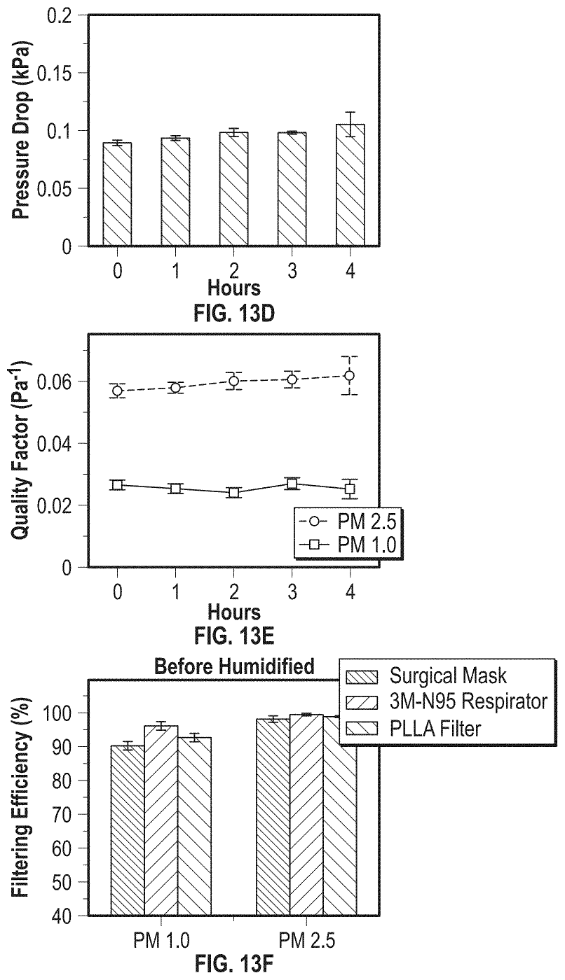

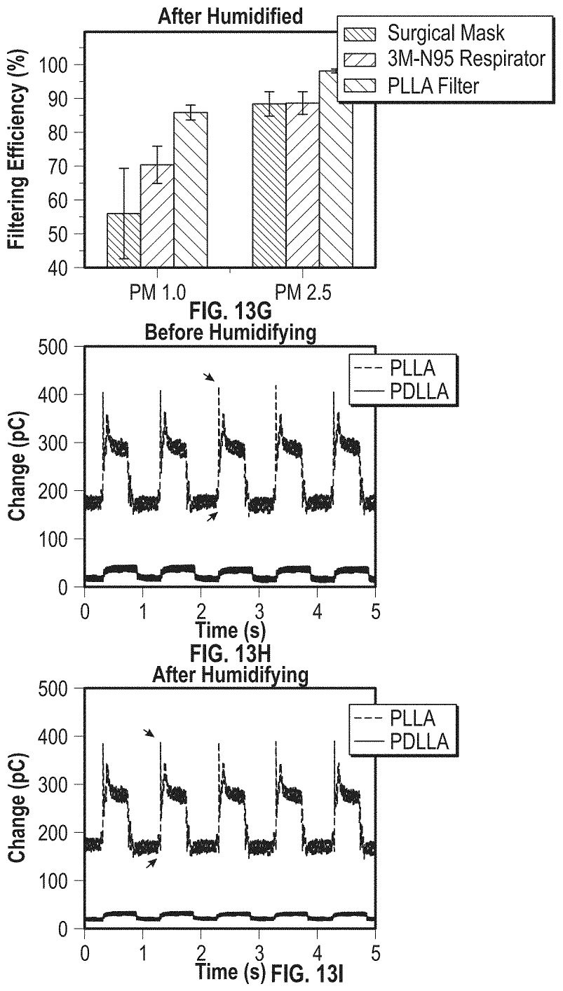

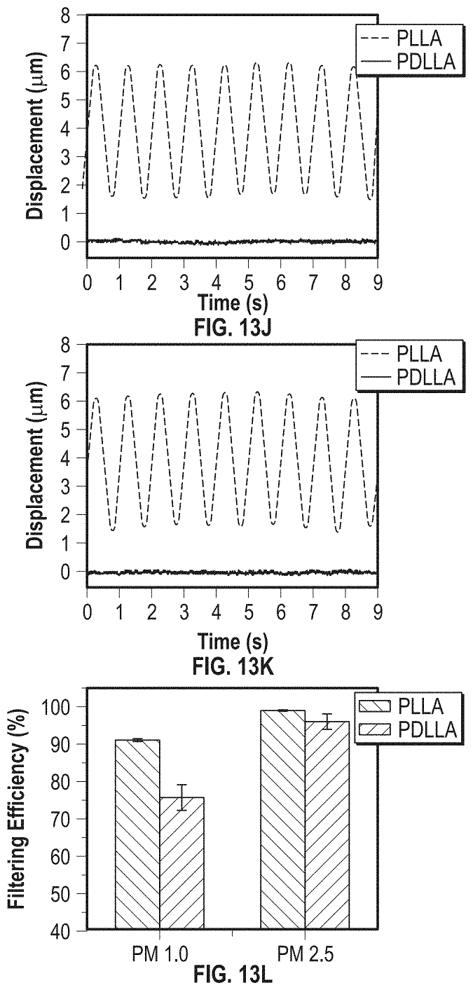

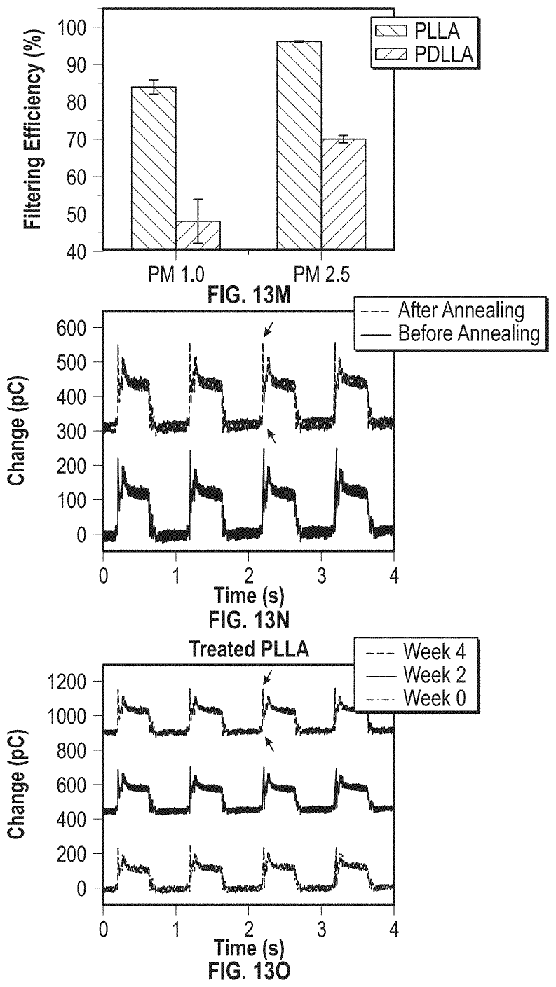

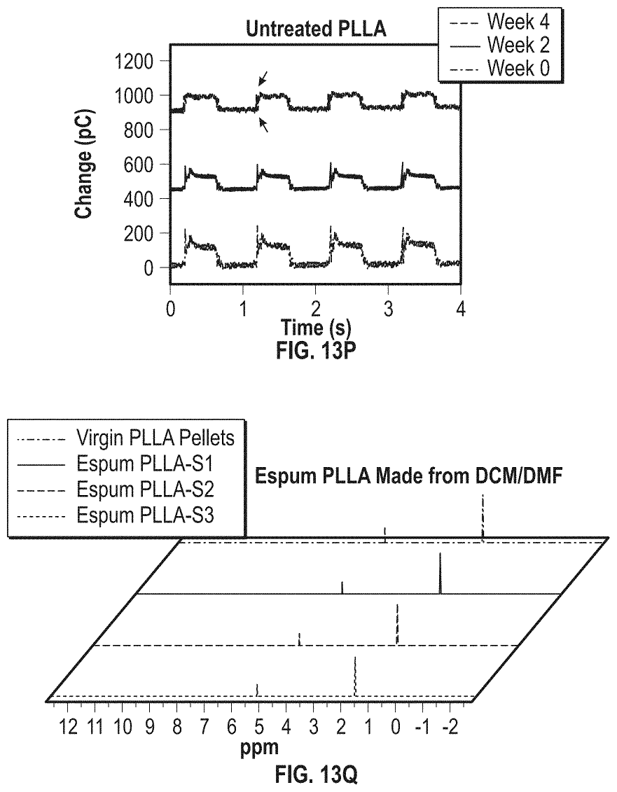

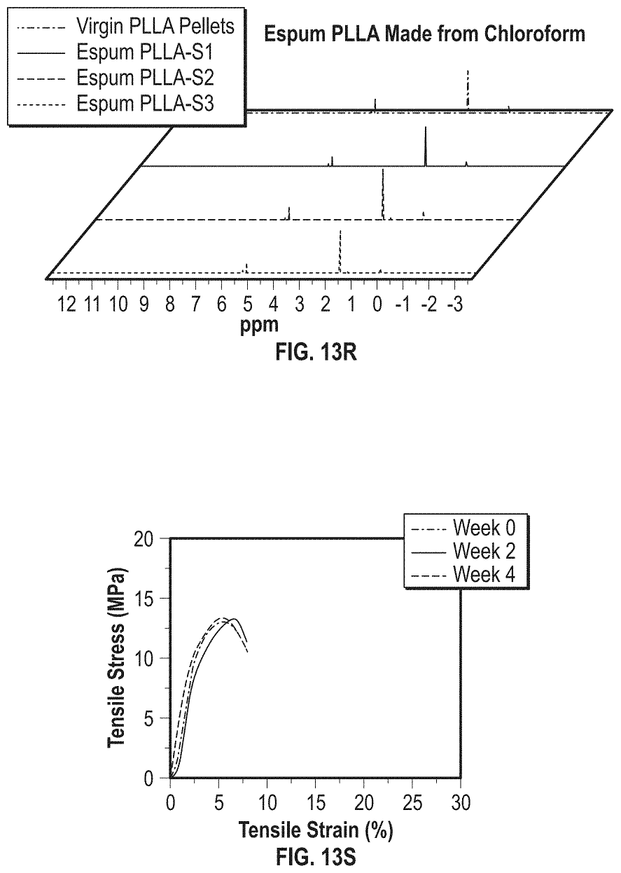

Commonly utilized face masks, such as the N95, KN95, and surgical masks, rely on their microporous structure (˜15 μm average pore size diameter) and electrostatic surface charges to filter out particulate matter from air passing through the mask. However, due to the reliance of these masks on electrostatic charges, their filtration efficiency has been shown to not be stable and reduced at elevated humidity levels, since the charge is screened and neutralized by ions in the environment. Specifically, these facemasks are susceptible to moisture from high humidity environments or human breath which contains abundant water molecules, forming an antistatic layer that dissipates charge. In contrast, the piezoelectric effect allows PLLA to be self-charged by an applied mechanical impact force, thus it always generates an output charge under air flow. This unique property of the PLLA piezoelectric nanofibers enables a great moisture resistance, compared to the commercial N95 mask filter and the surgical mask. This stable filtering efficiency of the PLLA nanofibers is illustrated in FIG. 13A. In this experiment, all the filtering materials (including PLLA nanofiber films, N95 filtering membranes and common surgical masks) were wetted by vigorously spraying them with water via a mist nebulizer. Initially, the filtering efficiency of each sample was measured at normal breathing rate (face velocity 5.83 cm/s). All of these samples were then wiped with a Kimwipe and air-dried before testing the filtering efficiency. The decrease in the filtering efficiency was computed as follows:

$$D \% = 100 \times ( 1 - \frac{{E}_{AW}}{{E}_{BW}} )$$

With E_{AW} and E_{Bw} representing the filtering efficiencies after and before wetting, respectively. It is apparent (from FIG. 13F-G) that the piezoelectric PLLA sample shows a negligible loss (about 0.8%) in regard to PM 2.5 removal, while a loss of 9.7% and 10.7% in filtering efficiency were observed for the surgical mask and N95 mask filter, respectively.

In addition, these filter samples were also challenged with PM 1.0. As demonstrated in the figure, while the N95 mask filter and the surgical masks suffered a significant loss in the filtering efficiency (26.8% and 38.1%, accordingly), the piezoelectric PLLA nanofiber mat experienced a minor reduction of 7.5% (FIGS. 13A, 13F, 13G).

Also, to verify the impact of the piezoelectricity on the minimal decline in PM filtration performance in a humid environment, the piezoelectric performance of the PLLA nanofiber mat was assessed through an impact test (i.e., generation of charge under applied dynamic force) and an actuation test (i.e., displacement under an applied electric field) before and after humidifying process. In the impact experiment, the piezoelectric response of the PLLA sample slightly decreased (˜15%) after water exposure due to the reduction of triboelectric effect in humid conditions (FIGS. 13H-I, arrows indicating peak of the output charge under the impact force). However, in the actuation experiment, where triboelectricity plays no part, the displacement of the PLLA nanofiber membranes was not affected after exposure to a humid environment (FIGS. 13J-K). To further validate the effect of piezoelectricity in the filtration performance of the material, the filtering efficiency of the non-piezoelectric PDLLA and piezoelectric PLLA samples were characterized before and after humidifying. As expected, the filtering performance of PDLLA dramatically dropped (from 75.7% to 48% for PM 1.0 removal) after water exposure while the drop was minimal for the piezoelectric PLLA (FIGS. 13L-M). Consequently, these results confirm the moisture resistance of the piezoelectric PLLA nanofiber films. It should be noted that the testing environment that was used in this test is harsher than the ambient humidity caused by sweating, coughing, and sneezing; hence, this piezoelectric PLLA filter should endure exhaled moisture and maintain its filtration performance for practical use in a facemask application.

One of the significant issues of the piezoelectric PLLA is the depolarization of the PLLA crystals over time which reduces the piezoelectric response of the membrane leading to the reduction in filtration performance. Here, through a suitable strategy for materials processing, the piezoelectric PLLA nanofiber mat can achieve an outstanding and long-lasting piezoelectric response as was previously reported for long-term use in implantable devices. As shown in FIG. 13N, the piezoelectric performance (i.e., output charge under applied force) of the PLLA nanofiber samples before and after the annealing process are comparable. However, when characterizing the piezoelectric response of the treated PLLA and untreated PLLA nanofibers over time (FIGS. 13O-P), a significant drop in piezoelectricity from the untreated sample was found, while the piezoelectric response of the treated PLLA remained stable. The arrows indicate the peak of the output charge under the impact force. It is also noteworthy that the solvents used to make the PLLA nanofibers are completely removed after the electrospinning and post-annealing process, and therefore present no risk to the mask user (FIGS. 13Q-R). Furthermore, as demonstrated in FIG. 13B, the treated piezoelectric PLLA nanofiber mat that underwent the full annealing process (i.e., two series of annealing and slowly cooling) possesses a stable filtering efficiency (100% retention after 8 weeks) in a regular room environment (see methods in the SI) due to the stable piezoelectric effect from material processing. On the contrary, the untreated (i.e., not annealed) samples rapidly and significantly lose their performance from 91.30% to 69.05% and from 97.6% to 90.6% for PM 1.0 and PM 2.5 filtering efficiency, respectively. Besides its filtration performance, the mechanical property of the treated PLLA nanofiber filter remains unchanged after 4 weeks (FIG. 13S). These results not only verify the crucial contribution of the piezoelectricity in enhancing and maintaining the filtration performance of the samples, but also illustrate the long-term stability of PLLA, which offers longer storage and usage time. In addition, to illustrate the durability of the PLLA nanofiber membrane, the film was characterized for continuous use over several hours. To do so, the samples were continuously exposed to the filtration test for 240 minutes. As described in FIGS. 13C-E, both the filtering efficiency and the air pressure differential slightly increase after 240 minutes, but they have comparable magnitudes. This minor increment could be due to the agglomeration of the PMs on the filter surfaces blocking the pores of the membranes. However, the quality factor does not vary significantly, which indicates that the PLLA filter can deliver a high filtration performance and excellent durability for a prolonged period of continuous use.

Bacteria Retention, Sterilizability and Reusability of the Piezoelectric PLLA Nanofiber Membrane.

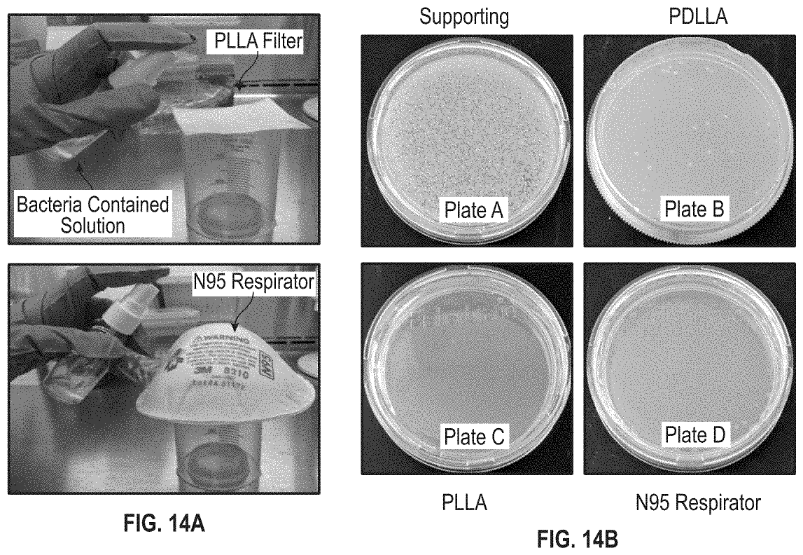

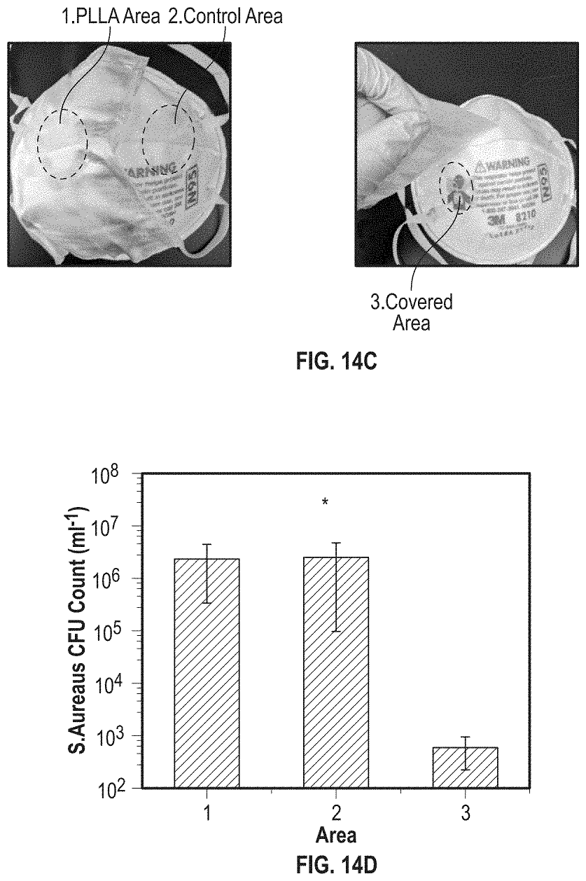

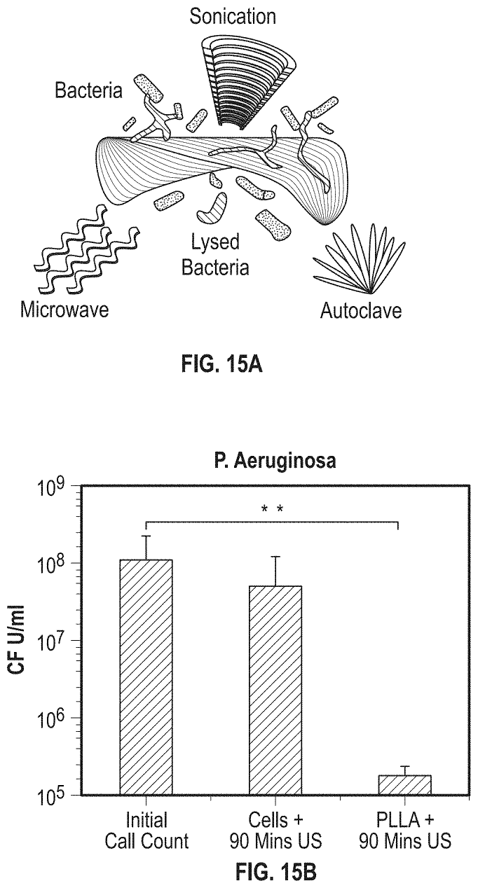

As mentioned, the SARS-COV-2 virus, for example, can easily transmit and spread through airborne particulate matter, causing severe global public health issues. As the proposed PLLA filter's objective is to prevent the spread and transmission of the virus, the ability to adsorb viruses/bacteria for the piezoelectric membranes were characterized. In the model study, the PLLA nanofiber air filter was challenged with Staphylococcus aureus (S. aureus)-containing aerosols generated by a mist nebulizer in order to mimic the aerosol particles from sneezing and coughing. In addition, the non-piezoelectric PDLLA and supporting layer were used as control samples and the commercial N95 respirator were used as a positive control to compare the bacteria retention performance of the piezoelectric PLLA nanofiber membranes. Specifically, the aqueous solution containing S. aureus was sprayed on the samples, which were positioned 10 cm above the lysogeny broth (LB) agar plates (FIG. 14A). The bacteria retention property of the samples was evaluated by the bacteria proliferation on the LB agar plates. As illustrated in FIG. 14B, bacterial particles penetrated through the supporting layer sample (plate A) and the PDLLA samples (plate B), whereas almost no bacteria were observed on the plate covered by the PLLA sample (plate C) and the N95 respirator (plate D). Additionally, to assess the bio-protection capacity of the PLLA filter quantitatively, the population of S. aureus captured on the challenged PLLA (FIG. 14C) were harvested and quantified. As can be seen in FIG. 14D, a large amount of S. aureus was trapped on the PLLA nanofiber membrane (Area 1), and this quantity is comparable to the one on the N95 respirator (Area 2); while a minimal number of bacteria (over 3 orders of magnitude difference) was detected on the covered area (Area 3). Hence, these results verify the bacteria retention property of the piezoelectric PLLA nanofiber membrane.

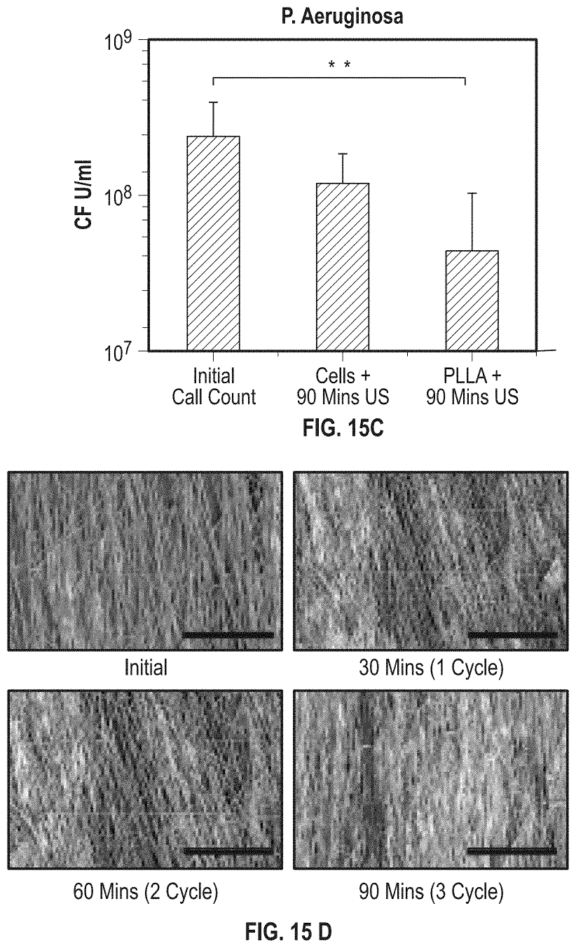

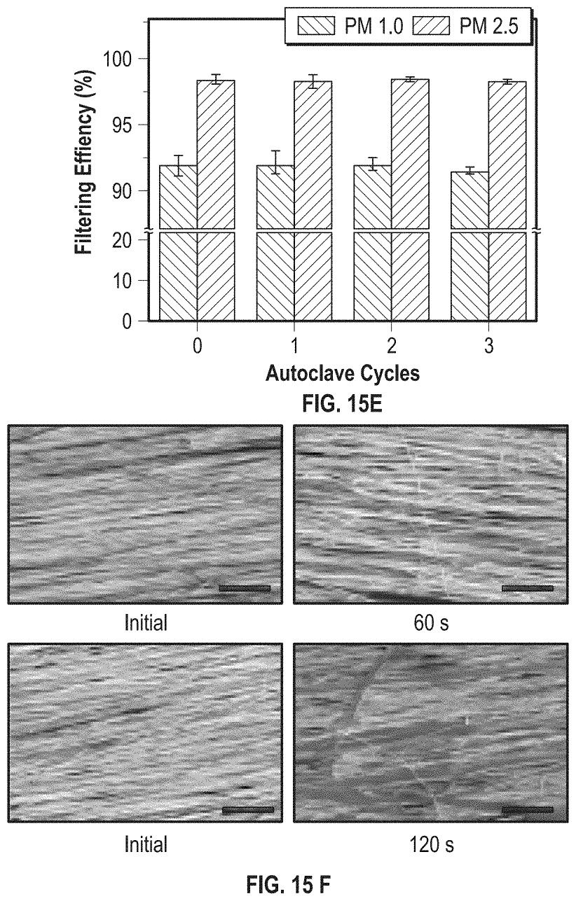

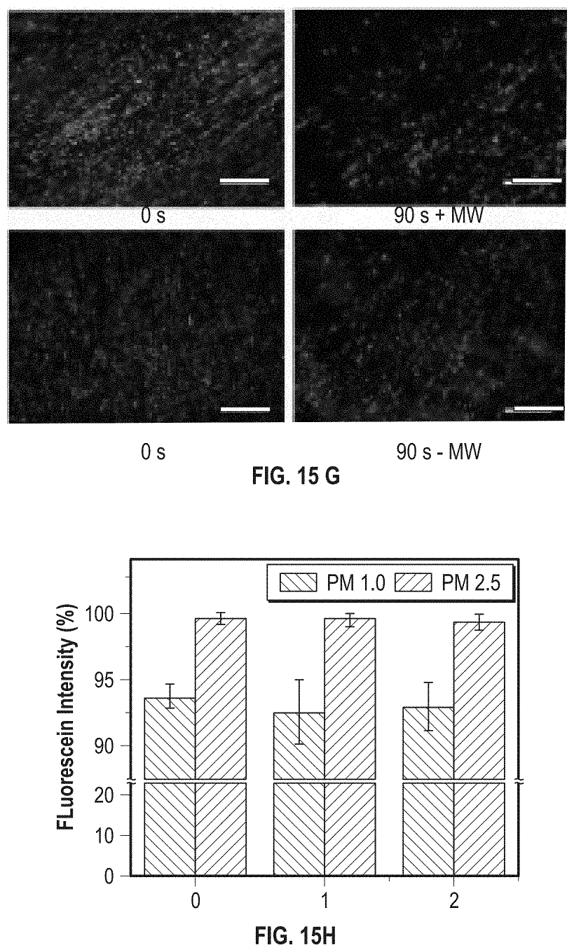

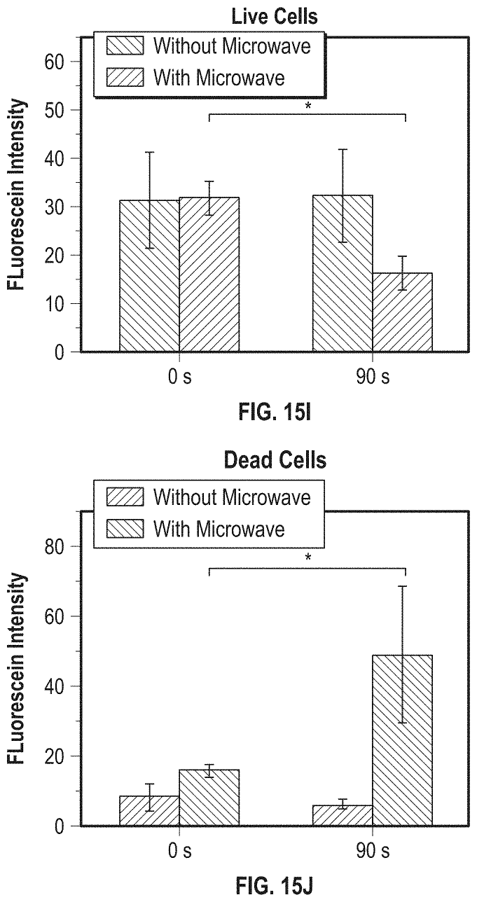

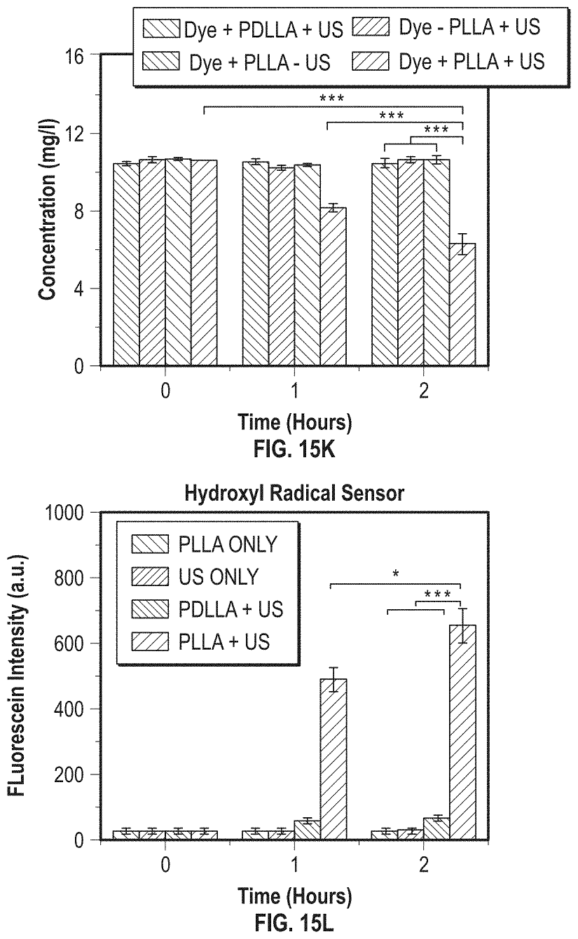

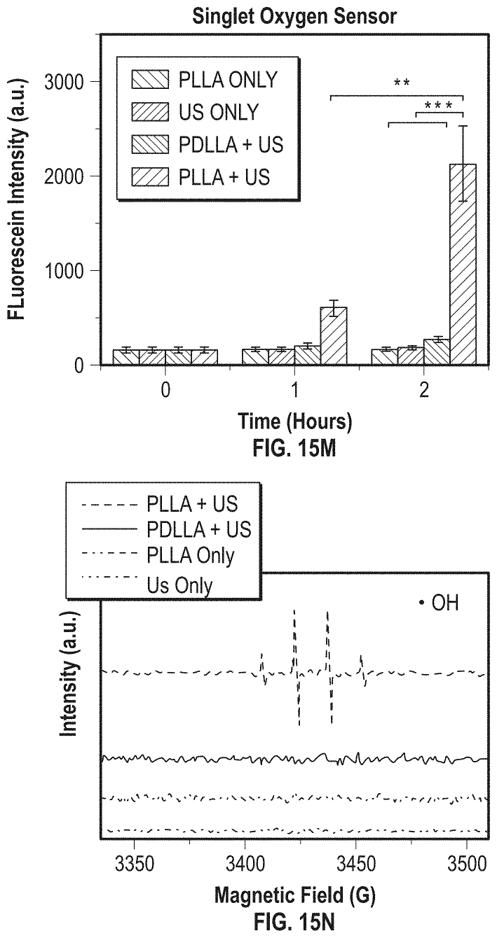

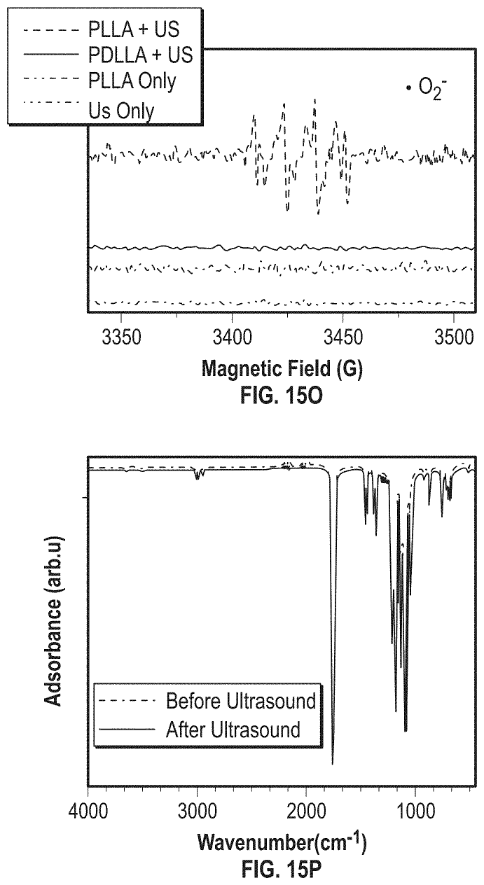

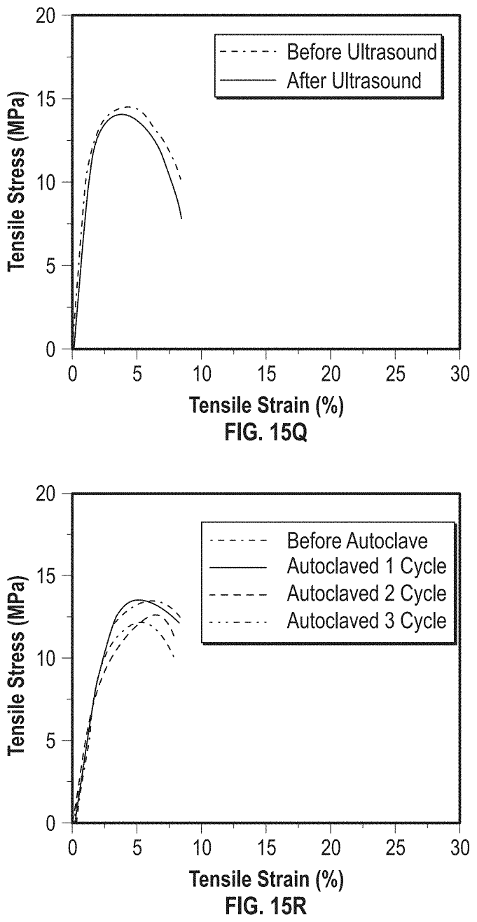

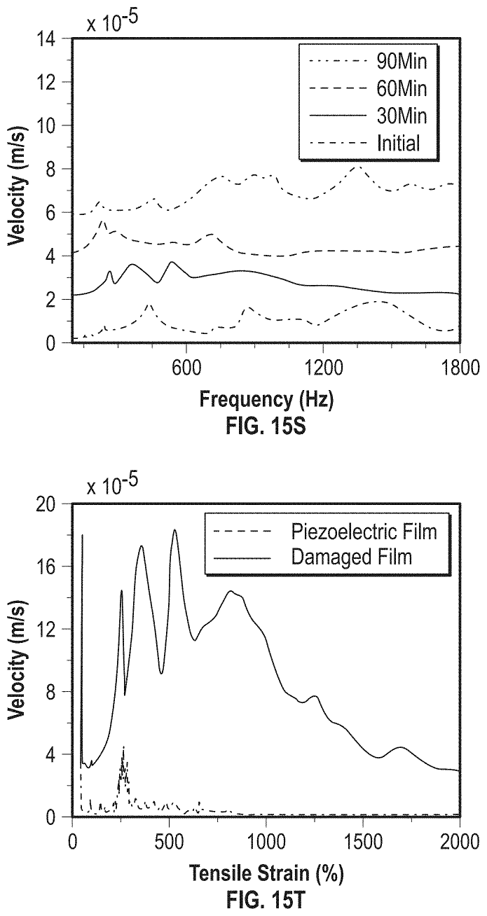

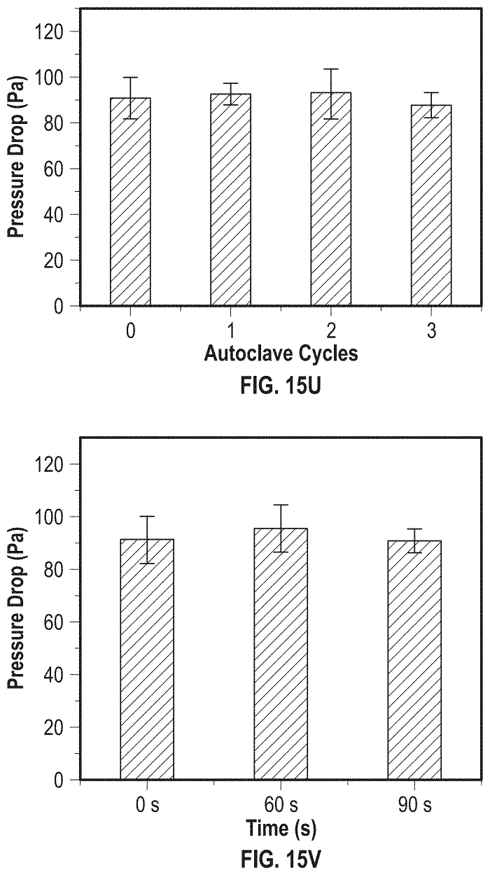

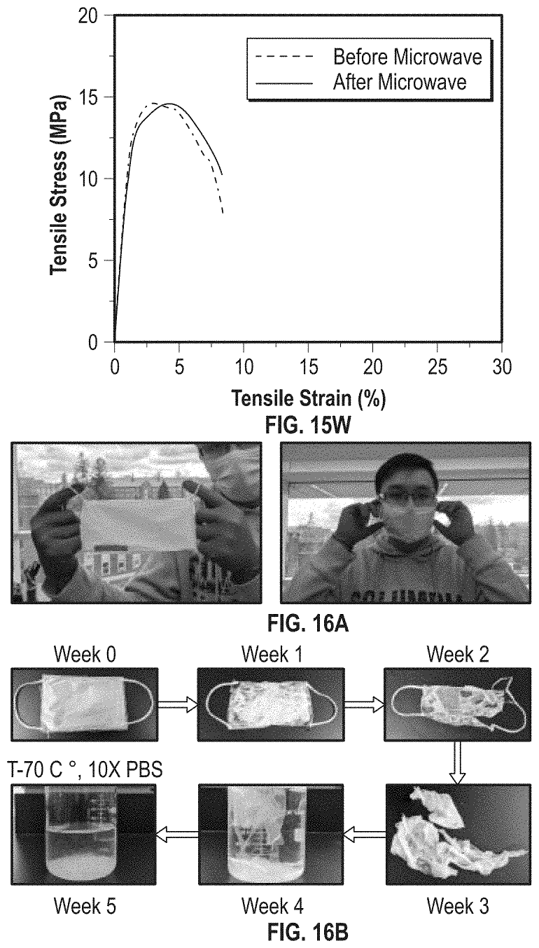

Apart from being vulnerable to high moisture, current conventional respirators (e.g., N95, KN95, and surgical masks) are not intended for multiple uses. Moreover, it is challenging to sterilize these masks after they are contaminated with viruses or bacteria, while avoiding a loss in their filtration performance. Here, the piezoelectric PLLA nanofiber filter can overcome these issues and can be reused via familiar decontamination or sterilization methods such as ultrasonic cleaning (using a household jewelry cleaner), autoclaving (steam sterilization), and microwaving (FIG. 15A). Notably, an approach called piezo-catalysis was used, which relies on mechanically-induced charges from piezoelectric materials to kill bacteria. Under mechanical stimulation (i.e., ultrasonic waves), piezoelectric nanofibers can generate piezoelectric charges that catalyze chemical reactions to produce a sufficient amount of anti-bacterial reactive oxygen species (ROS). To assess the piezoelectric PLLA nanofiber's capability to produce the ROS under applied ultrasound (US), the degradation of the Indigo Carmine solution was investigated, as the dye is known to be decomposed by the presence of ROS. The concentration of the dye in the phosphate buffer saline (PBS) exposed to the combination therapy of PLLA+US appears to have a statistically significant effect (p<0.001) when compared to the other control samples (i.e., non-piezoelectric or non-US). Additionally, the quantities of ROS can be tuned based on the duration of the applied US. This can be seen by the statistically significant difference (p<0.001) between the dye concentrations at 60 minutes and 120 minutes of ultrasonic treatment (FIG. 15K).

To further certify the presence of ROS generated by the piezoelectric PLLA combined with the US, a hydroxyl radical sensor and singlet oxygen sensor green, which emits green fluorescence when reacting with the hydroxyl radical (.OH) and singlet oxygen (102), respectively, were employed. As shown in FIGS. 15L-M, a remarkable fluorescein signal representing the existence of the ROS from the group of piezoelectric PLLA nanofiber membranes under ultrasound stimulation was detected. It is also worth noting that the intensity of green fluorescein increases with the ultrasound stimulation time. Moreover, electron paramagnetic resonance (EPR) spectroscopy using 5,5 dimethyl-1-pyrroline N-oxide (DMPO) as a spin trapper has been employed to validate the generation of .OH and superoxide anion (.O₂—), which are common reactive species for piezo-catalysis effect. The identification peaks of both DMPO-.OH and DMPO-.O₂— (FIGS. 15N-O) were only detected in the group of piezoelectric PLLA combined with the US while no peaks were observed from other control groups.

While the generation of ROS and the hydroxyl radical are promising, one question is that whether the presence of ROS in the solution could lead to the accelerated decomposition of the PLLA nanofiber mesh, making it non-reusable. To confirm the stability of the PLLA nanofiber membranes before and after US, Fourier-transform infrared spectroscopy (FTIR) was used to observe its molecular structure. After 90 minutes of ultrasonic stimulation, there is no obvious change in molecular structure of the PLLA molecule (FIG. 15P). The mechanical properties of the membranes before and after the sonication process was assessed and it was found that this sterilization procedure did not affect its mechanical properties (FIG. 15Q). Therefore, these results certify the possibility to re-use the piezoelectric PLLA nanofiber membranes.

Furthermore, in order to illustrate the antibacterial effects of the piezoelectric PLLA, the filter was submerged in an electrolytically conductive solution (phosphate-buffered saline (PBS)), with roughly 1×108 CFU/ml of P. aeruginosa (strain: Boston 41501) and S. aureus (strain: Wichita), and then exposed them in the sonication bath for 90 mins. As can be seen in FIGS. 15B-C, the piezoelectric PLLA combined with ultrasound stimulation has a statistically significant effect in killing P. aeruginosa (p<0.01) and S. aureus (p<0.01) bacteria. This result indicates the material's ability to lyse both gram-negative and gram-positive bacteria and thus, the material can be disinfected via an ultrasonic water bath.

Beside ultrasonic cleaning, the piezoelectric PLLA nanofibers can also be sterilized with an autoclave, which utilizes superheated steam to kill microorganisms and bacteria. As shown in FIG. 15D, the nanofiber films maintain their structure after 1 and 2 cycles of autoclaving; on the other hand, after 3 cycles, there are some fibers on the surface of the mats that have detached or broke. However, given that the macroscopic PLLA nanofiber meshes remain intact and their mechanical properties are comparable after three periods of autoclaving (30 minutes each) (FIG. 15R), it was determined that the PLLA nanofiber-based filter can be autoclaved for at least 90 minutes. To ensure that the PLLA nanofiber filter still exhibits its piezoelectricity, the piezoelectric effect of the PLLA filters after different autoclaving periods has been characterized by an actuation test using a laser scanning vibrometer to identify the vibration of the film under applied voltages. It appears that piezoelectric performance of the PLLA nanofiber still remains after the autoclave procedure (FIGS. 15S-T). Furthermore, the autoclaved piezoelectric PLLA samples showed little-to-no decrease in filtering efficiency as well as pressure drop (FIGS. 15E and 15U). The data clearly supports the use of an autoclave to sterilize the PLLA nanofiber mat without affecting the nanofiber performance.

For the ultrasonic or autoclaving methods, users can drop the PLLA-based face masks at common healthcare facilities to decontaminate the filtering mats while picking up new ones. However, for those who have limited access to these facilities, it would be a challenge for them to sterilize their facemask regularly. Microwaves have been reported to be able to neutralize viruses and bacteria, and thus, it can be used for sterilizing laboratory tools. Hence, the disinfection of the piezoelectric PLLA nanofiber filter was tested by utilizing a household microwave oven, with the intention that users can disinfect their mask conveniently at home. In this regard, the piezoelectric PLLA nanofiber membranes were wetted with deionized water, then dried before placing them in the microwave oven for 90 seconds. As illustrated in FIG. 15F, the fiber morphology of the PLLA nanofibers is stable after 90 seconds of microwaving which provided a sufficient time for the decontamination process (i.e., the ability to kill bacteria or cells on the PLLA material as seen in FIG. 15G). It also appears that this sterilization process does not affect the filtration performance (i.e., filtering efficiency, pressure drop, mechanical property) of the PLLA (FIGS. 15H, 15V, 15W). To quantify the disinfecting effect of the microwave method, cells (adipose-derived stem cells or ADSC) were seeded on the piezoelectric PLLA nanofiber films and microwaved them for 90 seconds. Then a live/dead assay was used to evaluate the cell lysing effect of microwaves. As demonstrated in FIG. 15G most of the cells have been killed after the microwave procedure. The fluorescein intensities of the live and dead cells before and after microwaving are represented in FIGS. 15I-J. A remarkable increase in dead cells was observed in the samples undergoing 90 seconds of microwaving. This result does not mean the microwave procedure can completely neutralize viruses and bacteria. However, it does indicate that microwave is a promising approach that enables an easy-to-use and accessible sterilization process for the piezoelectric PLLA nanofiber filter.

Biodegradability of the Piezoelectric PLLA Nanofibers Membrane.

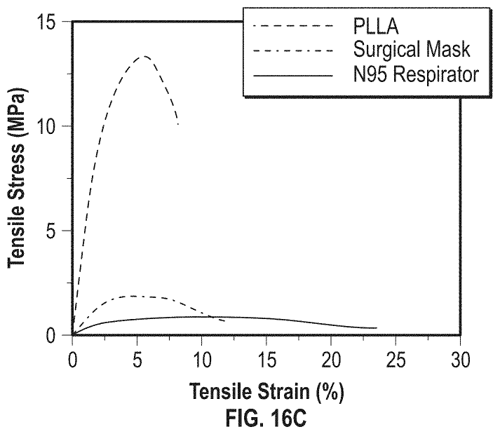

Besides the outstanding filtration performance and reusable capability, the PLLA nanofiber layers are also eco-friendly because of their long-term biodegradability, which can help prevent the plastic-waste crisis due to the enormous use of conventional non-degradable facemasks. To demonstrate this significant environmentally friendly property of the PLLA filter, the first prototype was made of a completely biodegradable piezoelectric facemask (FIG. 16A). In this design, the piezoelectric PLLA nanofiber filter layer was sandwiched between two supporting layers made of biodegradable PLLA nanofibers (with little-to-no piezoelectric effect), electro-spun at a very low (i.e., 300 rpm) collector drum speed. The wire loop was made from biodegradable natural rubbers. The facemask was assembled using an ultrasonic welding system (FIG. 1). PLLA has a long degradation time (about 2-3 years) which is beneficial to the longevity of the biodegradable face mask while still avoiding the problem of permanent plastic waste. Due to the long degradation lifetime of PLLA, to accelerate the mask's degradation for demonstrating its biodegradability, a highly concentrated buffer solution (PBS 10×, PH=7) and a high temperature of 70° C. were used. As shown in FIG. 16B, the facemask mostly degraded after five weeks under the accelerated degradation conditions. Additionally, the mechanical properties of the PLLA nanofiber membrane were characterized, as well as the filtering layers of commercially available facemasks (i.e., surgical masks, N95 respirators). As shown in FIG. 16C, the PLLA filter possesses a remarkably higher ultimate stress (up to 13.3 MPa), which overcomes the issue of low mechanical strength from most present nanofiber materials (usually <10 MPa). Hence, the PLLA nanofiber membrane can endure higher stress and is sturdier than the filtering layers of the surgical masks as well as the N95 respirators. The results clearly illustrate the ability to use the piezoelectric PLLA nanofiber membrane for creating a robust, highly effective, and environmentally friendly facemask.

### CONCLUSION

In summary, disclosed herein is a biodegradable, self-sustainable, moisture-resistant, highly effective, and reusable facemask filter based on a piezoelectric PLLA nanofibrous membrane. The PLLA nanofibers can always produce piezoelectric charge whenever there is an air flow (such as the one from human respiration), generating an electrostatic shielding layer to prevent the penetration of particles or water droplets which potentially carry viruses or bacteria. The piezoelectric effect improves the removal of PMs via an enhanced electrostatic/charge adsorption and enables the PLLA piezoelectric nanofiber filter to overcome the current issues of the conventional facemasks such as moisture susceptibility, non-degradability, and poor reusability. One of the embodiments of the PLLA filter samples (2 layers with a 6×6 holes on top layer) achieved excellent filtering efficiencies up to 91.48% and 99.21% for PM 1.0 and PM 2.5, respectively, while possessing an appropriate air pressure differential of 91 Pa (˜0.09% atmospheric pressure) for breathability. The PLLA nanofiber filter can also be sterilized using common tools such as an ultrasonic bath, autoclave, and microwave, potentially enabling users to conveniently disinfect and reuse the filter. Moreover, herein is demonstrated a completely biodegradable facemask prototype, made from the piezoelectric PLLA nanofiber membrane filter. Due to its long-term biodegradability and reusability, the piezoelectric PLLA nanofiber-based facemask could offer a key eco-friendly solution to the global problem of massive plastic waste generated by the constant and enormous use of traditional non-degradable facemasks or respirators during (and even after) the pandemic. As a result, the piezoelectric PLLA nanofiber filter and the biodegradable piezoelectric facemask, presented herein, could have a great impact not only on significantly preventing the transmission of highly infectious viruses and filtering polluted air but also on solving an important environmental crisis at the global scale.

The disclosed facemask overcomes known facemasks. For example, over the past few years, there have been several attempts to enhance the electrostatic filtering performance of filters. Electrically activated materials such as polyacrylonitrile (PAN), polyimide (PI), polyamide-66 (PA-66), poly(vinylidene fluoride) (PVDF) have been used to fabricate nanofiber-based air filters for effective PM 2.5 as well as PM 0.3 removal. Also, facemasks integrated with triboelectric nanogenerators for high-efficiency filtering have also been investigated. Furthermore, other materials such as metal-organic frameworks and/or polytetrafluoroethylene (PTFE), as well as carbon nanotubes have also been incorporated into a nanofiber-based filter to achieve higher electrostatic adsorption. In addition, a self-sustained, highly efficient, electrostatic spider-web-inspired network generator air filter made from electrospraying-netted PVDF was recently reported. Nevertheless, the filtering layers in all aforementioned approaches are made from non-degradable materials that do not decompose in landfills, thus still causing the environmental problem of non-degradable plastic trash. Moreover, the biocompatibility of these materials interacting with the human lung has not been certified. So far, to address the concern of plastic pollution, there have been several reports on ecofriendly membranes for efficient air filtration systems. Still, the problem of losing electrostatic charge in a humid environment for these filters has not been resolved. To address this issue, a biodegradable, multiuse, highly efficient Janus membrane filter exists that is made from electrospun chitosan nano-whiskers—coated in poly(butylene succinate)—which possessed a stable electrostatic charge in ambient moisture environments. Yet, the preparation procedure of the filter is tedious, and its long-term stability is questionable due to the rapid decomposition of chitosan. It is also challenging to sterilize these filters under harsh conditions such as high temperature and high pressure (e.g., using autoclave or microwave) for reusability.

Another strategy to obtain an enhanced and stable charge-adsorption effect is to utilize piezoelectric materials, a type of “smart” material that can generate electrical charge under applied pressure. Due to the displacement of atoms inside the piezoelectric crystals, piezoelectric charges can always be created whenever there is an applied pressure such as the pressure caused by airflow or impact of water droplets. Therefore, piezoelectric materials offer an excellent air-filtering platform with sustained charges to retain a high particle trapping performance (since there is always supplied energy from air flow). Unfortunately, most traditional piezoelectric materials are non-degradable, while some even contain toxic elements, such as lead in Lead Zirconate Titanate (PZT). In this regard, herein is reported stable and highly-functional piezoelectric nanofibers of PLLA (poly(L-lactic acid)), a biocompatible polymer with long-term biodegradability, which could be used to fabricate an environmentally friendly mask with excellent particle-trapping efficiency. Although other researchers have reported on some air filtering membranes (mostly non-piezoelectric) from the use of electrospun PLLA nanofibers, they (1) lacked an appropriate material processing approach to stabilize the nanomaterial for long-term repeated use, (2) could not sterilize the filter, and (3) did not utilize the shear piezoelectricity of PLLA for an optimal piezoelectric performance, leading to a modest piezoelectric constant (˜2.25 pC/N) which prevents maximal charge generation for a high trapping efficiency and small-particle removal performance. Here, in contrast, is disclosed a stable, reusable, self-sustaining, highly effective, and humidity-resistant air filtration membrane with excellent particle-removal efficiency and a long-term biodegradability based on highly controllable and stable piezoelectric PLLA nanofibers (FIG. 7A). Table 1 below is an overall comparison between the disclosed facemask filtering membrane and other available mask filtering membranes. The electrospun PLLA nanofiber filter can effectively remove particulates, especially ultrafine particles (up to 91% for PM 1.0), while providing a favorable pressure drop of around 91 Pa for human breathing (at normal breathing rate) due to the nanofiber structure and the sustainability of piezoelectric charges generated by aeolian vibrations which enhance particle adsorption. The filter has a long, stable filtration performance (up to 8 weeks) and good humidity resistance, which is demonstrated by a little reduction in the PM 2.5 filtration performance of the nanofiber membrane after exposure to moisture. Furthermore, herein is demonstrate that the PLLA filter is also reusable via common decontamination/sterilization processes (i.e., using an ultrasonic cleaning bath, autoclave, or microwave). Moreover, a prototype for the biodegradable PLLA nanofiber facemask was created and demonstrated the mask can decompose within 5 weeks (which often takes 2-3 years and even longer if stored appropriately for the PLLA) in an accelerated degradation environment with high temperature and concentrated saline buffer. Despite several achievements in the field, this work presents the first highly efficient, stable, moisture-resistant, sterilizable, and environmentally friendly air filtering membrane, based on piezoelectric PLLA nanofibers with long-term biodegradability.

TABLE 1

| | | | | Long- | | | |
|---|---|---|---|---|---|---|---|
| | | | Self- | term | Moisture | Steriliz- | Bio- |
| | Effi- | Pressure | Sustain- | Stabil- | Resis- | ability and | degrad- |
| Samples | ciency | drop | ability | ity | tance | Reusability | ability |
| Electrospun | 99.2% | 170 | v | v | x | N/A | x |
| PVDF | for PM | Pa | | | | | |
| | 2.5 | | | | | | |
| | 86.9% | | | | | | |
| | for PM | | | | | | |
| | 1.0 | | | | | | |
| Electrospun | 99.97% | 57 Pa | v | v | N/A | N/A | x |
| PVDF/PTFE | for | | | | | | |
| | PM 2.5 | | | | | | |
| Electro- | 99.03% | 88.5 | v | v | N/A | v | N/A |
| spraying- | for | Pa | | | | | |
| netting PVDF | PM 2.5 | | | | | | |
| | 98.33% | | | | | | |
| | PM 1.0 | | | | | | |
| Hydroxy- | 95% | 128 | x | v | N/A | v | x |
| apatite | for PM | Pa | | | | | |
| nanowires | 2.5 and | | | | | | |
| | PM 10 | | | | | | |
| Electrospun | 99.97% | 73 Pa | x | v | N/A | N/A | x |
| Polyimide | for PM | | | | | | |
| | 2.5 | | | | | | |
| Electrospun | 98.69% | 133 | x | v | v | N/A | x |
| polyacrylo- | for | Pa | | | | | |
| nitrile | PM 2.5 | | | | | | |
| Nano-fiber/net | 99.9% | below | x | N/A | N/A | N/A | x |
| Polyamide-66 | | 100 Pa | | | | | |
| Poly(vinyl | 99.99% | 90 Pa | x | N/A | N/A | v | v |
| alcohol) and | for PM | to 150 | | | | | |
| konjac | 0.3 | Pa | | | | | |
| glucoman | | | | | | | |
| loaded ZnO | | | | | | | |
| partilces | | | | | | | |
| Silk Nanofiber | 98.8% | 98 Pa | x | N/A | N/A | v | v |
| | for PM | | | | | | |
| | 2.5 | | | | | | |
| Chitosan | 98.3% | 59 Pa | x | v | v | N/A | v |
| Nanowhisker | for PM | | | | | | |
| coated | 2.5 | | | | | | |
| electrospun | 91% | | | | | | |
| PBS | for PM | | | | | | |
| | 1.0 | | | | | | |
| Electrospun | 99.3% | 95 Pa | v | N/A | N/A | N/A | v |
| PLLA | for PM | | | | | | |
| | 2.5 | | | | | | |
| | N/A for | | | | | | |
| | PM 1.0 | | | | | | |
| Commercial | above | from | x | x | x | v | x |
| N95 | 95% | 20-80 | | | | | |
| | | Pa | | | | | |
| Commercial | varies | from | x | x | x | x | x |
| Surgical Mask | from 25- | 20-40 | | | | | |
| | 90% | Pa | | | | | |
| Present | 99.3% | from | v | v | v | v | v |
| Piezoelectric | for PM | 91-120 | | | | | |
| Electrospun | 2.5 | Pa | | | | | |
| PLLA | 93% | | | | | | |
| | for PM | | | | | | |
| | 1.0 | | | | | | |

Although the discussion herein is directed to facepiece respirators, it should be understood that the same or similar structure and methods discussed herein may be used to form non-medical masks.

Various features and advantages of the invention are set forth in the following claims.

What is claimed is:

1. A facepiece respirator configured to be worn by a user, the facepiece respirator comprising:
a first layer being positionable adjacent the user;
a second layer coupled to the first layer; and
a filtering layer positioned between the first layer and the second layer, the filtering layer including a piezoelectric film.

2. The facepiece respirator of claim 1, wherein each of the first and second layers includes a biodegradable medical polymer.

3. The facepiece respirator of claim 2, wherein the biodegradable medical polymer includes at least one of poly-(l-lactic acid) (PLLA), polycaprolactone (PCL), poly(lactic acid) (PLA), poly(lactic-co-glycolic acid) (PLGA), polyurethane (PUR), and polyhydroxybutyrate (PHB) poly-(d-lactic acid) (PDLA), chitosan, chitin, poly(γ-benzyl-L-glutamate) (PBLG) or their combination with nanofiller dopants such as amino acid crystals (e.g. glycine) or cellulose nanocrystals.

4. The facepiece respirator of claim 1, wherein the piezoelectric film is biodegradable.

5. The facepiece respirator of claim 1, wherein the piezoelectric film is a poly-(l-lactic acid) (PLLA) nanofiber mat.

6. The facepiece respirator of claim 1, further comprising at least one loop coupled to the first layer or the second layer or the filtering layer.

7. The facepiece respirator of claim 6, wherein the at least one loop is formed from a biodegradable material.

8. The facepiece respirator of claim 1, wherein each of the first layer, second layer, and filtering layer is capable of being decontaminated at least once via an autoclave, microwave, or an ultrasonic cleaning bath.

9. The facepiece respirator of claim 1, wherein the piezoelectric film is a first piezoelectric film and wherein the filtering layer further comprises a second piezoelectric film.

10. The facepiece respirator of claim 1, wherein the piezoelectric film includes one or more holes extending therethrough.

11. A facepiece respirator configured to be worn by a user, the facepiece respirator comprising:
a first layer positionable adjacent the user;
a second layer coupled to the first layer; and
a filtering layer positioned between the first layer and the second layer, the filtering layer including an electrospun piezoelectric film,
wherein each of the first layer, the second layer, and the filtering layer comprise a sterilizable and biodegradable material.

12. The facepiece respirator of claim 11, further comprising at least one loop coupled to the first layer or the second layer or the filtering layer, the loop configured to position the facepiece respirator adjacent a nose and mouth of the user, the at least one loop comprising a sterilizable and biodegradable material.

13. The facepiece respirator of claim 11, wherein the sterilizable and biodegradable material includes poly-(l-lactic acid) (PLLA).

14. The facepiece respirator of claim 11, wherein the electrospun piezoelectric film is a first electrospun piezoelectric film and wherein the filtering layer further comprises a second electrospun piezoelectric film formed from a sterilizable and biodegradable material, the sterilizable and biodegradable material of each of the first piezoelectric film and the second piezoelectric film include a poly-(l-lactic acid) (PLLA) nanofiber mat, at least one of the first piezoelectric film and the second piezoelectric film including one or more holes extending therethrough.

15. A method of manufacturing a facepiece respirator, the method comprising:
forming a piezoelectric film by electrospinning a first material solution dissolved in a solvent to form a nanofiber mat;
applying heat to the nanofiber mat; and
positioning the piezoelectric film between a first layer and a second layer, the first layer being positionable adjacent to a user.

16. The method of claim 15, wherein forming the piezoelectric film by electrospinning a first material solution dissolved in a mixture to form a nanofiber mat includes
applying a voltage to the solution to form an electrified solution; and
applying the electrified solution to a drum rotating at a speed of between 500 rpm and 4,500 rpm.

17. The method of claim 15, wherein applying heat to the nanofiber mat includes
annealing the nanofiber mat at a first temperature for a first time period; and
annealing the nanofiber mat at a second temperature for a second period of time.

18. The method of claim 15, wherein the first material solution is a 4% w/v solution poly-(l-lactic acid) (PLLA) dissolved in either a 1:4 mixture of N,N-Dimethylformamide (DMF) and dichloromethane (DCM) or Chloroform.

19. The method of claim 15, wherein forming the piezoelectric film includes, after applying heat to the nanofiber mat, cutting the nanofiber mat to harvest a shear piezoelectric signal of the piezoelectric film.

20. The method of claim 19, wherein the piezoelectric film is a first piezoelectric film and the nanofiber mat is a first nanofiber mat and wherein the method further comprises
forming a second piezoelectric film by electrospinning the first material solution dissolved in the solvent to form a second nanofiber mat;
applying heat to the second nanofiber mat;
cutting the piezoelectric film to harvest a shear piezoelectric signal of the piezoelectric film;
removing material from the second piezoelectric film to create holes extending therethrough; and
positioning the first piezoelectric film adjacent to the second piezoelectric film.
